SEQUENCE LISTING (9 sequences)

SEQ ID NO: 1 - DNA, 30 bp, Artificial
  PCR primer.
tgccagaggattatcctgatcagttcgatg

SEQ ID NO: 2 - DNA, 30 bp, Artificial
  PCR primer.
ttgtcgtacgttgtctcagctgcatcgcaa

SEQ ID NO: 3 - DNA, 23 bp, Artificial
  PCR primer.
tcatgaagtgtgacgttgacatc

SEQ ID NO: 4 - DNA, 23 bp, Artificial
  PCR primer.
agaagcatttgcggtggacgatg

SEQ ID NO: 5 - DNA, 26 bp, Artificial
  PCR primer.
tgaaggtcggtgtcaacggatttggc

SEQ ID NO: 6 - DNA, 24 bp, Artificial
  PCR primer.
catgtaggccatgaggtccaccac

SEQ ID NO: 7 - protein, 11 aa, Artificial
  Synthetic peptide.
YGRKKRRQRRR

SEQ ID NO: 8 - DNA, 24 bp, Artificial
  PCR primer.
acactctggatctagacttccagg

SEQ ID NO: 9 - DNA, 24 bp, Artificial
  PCR primer.
aggcaagtcagcctcttcttgtag

(19) United States
(12) Patent Application Publication
(10) Pub. No.: US 2005/0203011 A1
(43) Pub. Date: Sep. 15, 2005

(54) Mitigating symptoms and behaviors of substance abuse by modulating GDNF or BDNF pathway activity

(75) Inventor: Dorit Ron, San Francisco, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/946,625

(22) Filed: Sep. 20, 2004

Related U.S. Application Data
(60) Provisional application No. 60/504,083, filed on Sep. 19, 2003.
(60) Provisional application No. 60/505,545, filed on Sep. 23, 2003.

(51) Int. Cl. C12Q 1/68; G01N 33/53; G01N 33/567; C12Q 1/48; A61K 38/12

(52) U.S. Cl. 514/12; 435/15; 435/7.2; 435/6

(57) ABSTRACT

This invention pertains to the discovery that brain-derived neurotrophic factor (BDNF) and its associated signaling pathway is involved in reversing and/or counteracting neuroadaptations within the mesolimbic system that contribute to the development and/or maintenance of addiction (e.g. alcohol addiction). This invention also pertains to the discovery that ibogaine activity is mediated by changes in mRNA expression of GDNF. Thus, in certain embodiments, this inventioni provides a method of mitigating one or more symptoms of substance abuse in a mammal by increasing the expression or activity of GDNF, BDNF, RACK1, and/or the dopamine D3 receptor (D3R) in said mammal.

### CROSS-REFERENCE TO RELATED APPLICATIONS

This application claims priority to and benefit of U.S. Ser. No. 60/504,083, filed on Sep. 19, 2003, and U.S. Ser. No. 60/505,545, filed on Sep. 23, 2003, both of which are incorporated herein by reference in their entirety for all purposes.

### STATEMENT AS TO RIGHTS TO INVENTIONS MADE UNDER FEDERALLY SPONSORED RESEARCH AND DEVELOPMENT

This work was supported, in part, by Grant # DAMD17-0110802 from the Department of the Army. The Government of the United States of America may have certain rights in this invention.

### FIELD OF THE INVENTION

This invention pertains to the field of neurobiology. In particular this invention pertains to the activity of the GDNF pathway and the BDNF pathway with respect to the response of an organism to substances of abuse.

### BACKGROUND OF THE INVENTION

The development of alcohol addiction depends upon a complex interplay of genetics, other biological factors, and social factors (Leshner (1997) Science 278: 45-47; Robbins and Everitt (1999) Nature, 398: 567570). Mechanistically, alcohol addiction is thought to depend upon molecular and cellular adaptations in the brain that result from chronic excessive alcohol intake (Fitzgerald and Nestler (1995) Clin. Neurosci. 3: 165-173; Nestler (2001) Nat. Rev. Neurosci., 2: 119-128). While prolonged, excessive consumption of alcohol can lead to the addictive phenotypes of tolerance, dependence, withdrawal, and relapse, moderate social drinking does not typically result in addiction. What holds moderate drinkers in check? An intriguing possibility is the existence of homeostatic protective mechanisms that prevent or delay the expression of the biochemical neuroadaptations that lead to the phenotypes associated with addiction. If these protective mechanisms do not function properly, then the neuroadaptations that underlie addiction may occur.

One candidate for such a homeostatic agent is the brain-derived neurotrophic factor (BDNF). BDNF belongs to the nerve growth factor (NGF) family and is an important mediator of neuronal survival. More recently, BDNF was found to play important roles in neurotransmitter release, synaptic plasticity and axonal and dendritic morphology (for review, see Chao (2003) Nat. Rev. Neurosci. 4: 299-309). Importantly, alteration in BDNF action modulates several behaviors associated with drugs of abuse (Hall et al. (2003) Neuropsychopharmacology 28: 1485-1490; Hensler et al. (2003) J. Neurochem. 85: 1139-1147; Horger et al. (1999) J. Neurosci. 19: 4110-4122), and studies have implicated BDNF in the reversal of some of the molecular and behavioral neuroadaptations within the mesolimbic circuitry to drugs of abuse. For example, infusion of BDNF into the ventral tegmental area (VTA) blocks the ability of morphine or cocaine to increase levels of tyrosine hydroxylase and the activity of cAMP-dependent protein kinase (PKA) (Berhow et al. (1995) Neuroscience 68: 969-979) and extracellular signal-regulated kinase 1 (Berhow et al. (1996) J. Neurosci. 16: 4707-4715) in this brain region. Additionally, BDNF has been shown to induce the expression of the dopamine D3 receptor (D3R), neuropeptide Y, and dynorphin in the striatum/nucleus accumbens (NAc) (Croll et al. (1994) Eur. J. Neurosci. 6: 1343-1353; Guillin et al. (2001) Nature 411: 8689), and an increase in the expression and/or function of each of these gene products attenuates the effects of addictive drugs whereas a decrease potentiates the effects of drugs of abuse (Carlezon et al. (1998) Science 282: 2272-2275; Thiele et al. (1998) Nature 396: 366-369; Pilla et al. (1999) Nature 400: 371-375; Xu et al. (1997) Neuron 19: 837-848).

Few medications have proven to be efficacious for treating disease states of drug addiction and dependence. Ibogaine, a natural alkaloid extracted from the root bark of the African shrub, Tabernanthe Iboga, has attracted attention due to its reported ability to reverse addiction to multiple drugs of abuse including alcohol (Popik et al. (1995) Pharmacol. Rev., 47: 235-253; Mash et al. (1998) Ann. N. Y. Acad. Sci., 844: 274-292; Vastag (2002) JAMA, 288: 3096, 3099-3101). Human anecdotal reports claim that a single administration of ibogaine reduces craving for opiates and cocaine for extended periods of time, and reduces opiate withdrawal symptoms (Sheppard (1994) J. Subst. Abuse Treat. 11: 379-385; Mash et al. (1998) Ann. N. Y. Acad. Sci., 844: 274-292; Alper et al. (1999) Am. J. Addict., 8: 234-242). Studies also suggest that ibogaine attenuates drug- and ethanol-induced behaviors in rodents. For example, ibogaine reduces operant self-administration of heroin in rats, as well as naloxone-precipitated withdrawal in morphine-dependent rats (Glick et al. (1992) Neuropharmacology 31: 497-500; Dworkin et al. (1995) Psychopharnacology (Berl) 117: 257-261). Administration of ibogaine decreases cocaine-induced locomotor activity and reduces cocaine self-administration in rats (Cappendijk and Dzoljic (1993) Eur. J. Pharmacol. 241: 261-265) and mice (Sershen et al. (1994) Pharmacol. Biochem. Behav. 47: 13-19). Rezvani et al. reported that ibogaine reduces ethanol self-administration in an alcohol-preferring line of rats (Rezvani et al. (1995) Pharmacol. Biochem. Behav., 52: 615-620), however, the effects of ibogaine have not been tested in an operant procedure in which oral ethanol reinforces lever press behavior.

Despite its attractive properties, ibogaine is not approved as a medication to treat addiction due to induction of side effects such as hallucinations. In addition, ibogaine causes degeneration of cerebellar Purkinje cells (O'Hearn and Molliver (1993) Neuroscience 55: 303-310; O'Hearn and Molliver M E (1997) J. Neurosci., 17: 8828-8841), whole body tremors, and ataxia (Glick et al. (1992) Neuropharmacology 31: 497-500; O'Hearn and Molliver (1993) Neuroscience 55: 303-310) in rats.

All drugs of abuse including alcohol activate the “reward” neurocircuitry (Koob et al. (1998) Neuron 21: 467-476; Spanagel and Weiss (1999) Trends Neurosci., 22: 521-527). One of the major brain regions in this pathway is the VTA. The VTA is the brain site where many biochemical neuroadaptations induced by repeated drugs of abuse, including ethanol, have been observed. For example, long-term ethanol exposure increases levels of tyrosine hydroxylase (TH), glial fibrillary acidic protein (GFAP), and the NR1 subunit of the NMDA receptor, and decreases levels of neurofilament (NF) protein and the a1 subunit of the GABAA receptor in the VTA (Beitner and Nestler (1991) J. Neurochem., 57: 344-347; Vrana et al. (1993) J. Neurochem. 61: 2262-2268; Ortiz et al. (1995) Synapse 21: 289-298; Charlton et al. (1997) J. Neurochem. 68: 121127). Furthermore, several studies suggest that the VTA may also be the site of action for reversal of the neuroadaptations that lead to the development of addiction. For example, infusion of the brain derived neurotrophic factor (BDNF) into the VTA reversed morphine-induced elevations of TH (Berhow et al. (1995) Neuroscience 68: 969-979). In addition, Messer at al (2000) reported that infusion of the glial cell line-derived neurotrophic factor (GDNF) into the VTA dose-dependently reversed the increase in TH immunoreactivity observed after morphine injection. Furthermore, chronic cocaine and chronic morphine exposure decreased the levels of phosphorylation, and thus activation, of GDNF in the VTA, and intra-VTA GDNF treatment blocked the behavioral effects of repeated exposure to cocaine (Messer et al. (2000) Neuron 26: 247-257). Thus, we postulated that the VTA, may be, at least in part, ibogaine's site of action.

### SUMMARY OF THE INVENTION

In one embodiment, this invention pertains to the discovery that brain-derived neurotrophic factor (BDNF) and its associated signaling pathway is involved in reversing and/or counteracting neuroadaptations within the mesolimbic system that contribute to the development and/or maintenance of addiction (e.g. alcohol addiction). In particular, we demonstrate that BDNF reduces the behavioral effects of ethanol, including consumption, in rodents. We found that decreases in BDNF levels or signaling results in increased behavioral responses to ethanol whereas increases in the levels of BDNF, mediated by the scaffolding protein RACK1, attenuates these behaviors. We also found that acute exposure of cultured neurons to ethanol leads to increased levels of BDNF mRNA via RACK1. Importantly, voluntary ethanol consumption also leads to increased levels of BDNF expression. Taken together, these findings indicate that RACK1 and BDNF are part of a homeostatic pathway that opposes adaptations that maintain addiction.

In another embodiment, this invention also pertains to the discovery that ibogaine activity is mediated by changes in mRNA expression of GDNF. In particular, it was observed that ibogaine decreased ethanol intake by rats in 2-bottle choice and operant self-administration paradigms. Ibogaine also reduced operant self-administration of ethanol in a reinstatement model. Using a conditioned place preference (CPP) paradigm, we found that ibogaine was not rewarding or aversive, nor did it alter the rewarding properties of ethanol. Microinjection of ibogaine into the ventral tegmental area (VTA) reduced self-administration of ethanol, and systemic administration of ibogaine increased the expression of the glial cell line-derived neurotrophic factor (GDNF) in a midbrain region that includes the VTA. In dopaminergic neuron-like SHSY5Y cells, ibogaine treatment upregulated the GDNF pathway, and reversed ethanol's effect on tyrosine hydroxylase (TH) protein levels via a GDNF-dependent mechanism. Finally, the ibogaine-mediated decrease in ethanol self-administration was reduced by intra-vta delivery of anti-GDNF neutralizing antibodies. Together, these results indicate that GDNF in the VTA mediates the action of ibogaine on ethanol consumption.

These findings indicate that the BDNF and GDNF pathways provide good targets for screening for agents that mediate one or more symptoms associated with substance abuse and upregulating expression of components of these pathways and/or pathway activity is an effective method of mitigating one or more symptoms of substance abuse or withdrawal therefrom.

Thus, in one embodiment, this invention provides a method of identifying an agent that mitigates one or more symptoms of substance abuse. The method typically involves contacting a cell or tissue with a test agent; and determining whether or not there is an increase in expression or activity of a GDNF pathway component, where an increase in expression or activity of a GDNF pathway or component, as compared to a control, indicates that the agent is an agent that mitigates a symptom of substance abuse. In certain embodiments the GDNF pathway component is selected from the group consisting of GDNF, GFRα1, and RET. In certain embodiments the determining comprises determining the association between GDNF and GFRα1 and/or the phosphorylation of RET. In certain embodiments the determining comprises determining the expression level of a component selected from the group consisting of GDNF, GFRα1, or RET. In certain embodiments is a nerve cell or tissue, a cell in a brain tissue preparation, a cell in culture (e.g., an SHSY5Y cell), etc. In various embodiments the determining comprises a nucleic acid hybridization to determine an mRNA level (e.g. of an mRNA encoding a component of a GDNF pathway). Suitable detection methods include but are not limited to a Northern blot, a Southern blot using DNA derived from an RNA encoding a protein in a GDNF pathway, an array hybridization, an affinity chromatography, an RT-PCR using an RNA encoding a protein in a GDNF pathway, and an in situ hybridization. In certain embodiments the detecting comprises detecting a protein in a GDNF pathway (e.g., via capillary electrophoresis, a Western blot, mass spectroscopy, ELISA, immunochromatography, immunohistochemistry, and the like). In certain embodiments the control comprises a cell contacted with the test agent at a lower concentration or a cell or tissue not contacted with the test agent at all. In certain embodiments the test agent is not an antibody, and/or not a protein, and/or not a nucleic acid. In certain embodiments the test agent is a small organic molecule. In various embodiments the test agent is contacted to a cell containing the component (e.g. a cell is cultured ex vivo) and/or administered to an animal comprising a cell containing the component.

This invention also provides a method of mitigating one or more symptoms (e.g., intake (self-administration) of the substance of abuse, preference for the substance of abuse, relapse, a symptom of withdrawal, etc.) of substance abuse in a mammal. The method typically involves increasing the level, expression, or activity of GDNF and/or a GDNF pathway in the mammal. In certain embodiments the method involves increasing the phosphorylation of RET. In certain embodiments the method does not comprise administering ibogaine. In certain embodiments the method comprises administering an ibogaine analogue. In certain embodiments the method comprises increasing the expression or activity of GFRα1. In certain embodiments the method comprises increasing the phosphorylation of RET. In certain embodiments the method comprises administering GDNF and/or a GDNF mimetic to the mammal.

In still another embodiment, this invention provides a method of mitigating one or more symptoms of substance abuse (e.g., intake (self-administration) of the substance of abuse, preference for the substance of abuse, relapse, a symptom of withdrawal, etc.) in a mammal. The method typically involves increasing the expression or activity of a BDNF pathway and/or BDNF, RACK1, and/or the dopamine D3 receptor (D3R) in the mammal. In certain embodiments the method comprises increasing the expression or activity of BDNF. In certain embodiments method comprises increasing the expression or activity of RACK1. In certain embodiments comprises increasing the expression or activity of the dopamine D3 receptor. In certain embodiments the method involves increasing the phosphorylation of TrkB. In certain embodiments the method comprises administering RAC1 or an analogue or mimetic thereof. In certain embodiments RAC1 is administered as a fusion protein tat-RAC1.

This invention also provides a method of identifying an agent that mitigates one or more symptoms of substance abuse. The method typically involves contacting a cell or tissue with a test agent; and determining whether or not there is an increase in increase in expression or activity of a component of a BDNF pathway, where an increase in expression or activity of a component of a BDNF pathway, as compared to a control, indicates that the agent is an agent that mitigates a symptom of substance abuse. In certain embodiments the determining comprises determining an increase in expression or activity of BDNF and/or RACK1. In certain embodiments the cell or tissue is a nerve cell or tissue, a cell in a brain slice preparation, a cell in culture, a cell in vivo, and the like. In certain embodiments the determining comprises determining the level of phosphorylation of TrkB. In various embodiments the determining comprises a nucleic acid hybridization to determine an mRNA level of a BDNF pathway component (e.g. BNDF, RACK1, TrkB, etc.). In certain embodiments the detecting comprises a method selected from the group consisting of a Northern blot, a Southern blot using DNA derived from an RNA encoding a protein in BDNF pathway, an array hybridization, an affinity chromatography, and an in situ hybridization. The detecting can comprise detecting a protein in a BDNF pathway (e.g., via capillary electrophoresis, a Western blot, mass spectroscopy, ELISA, immunochromatography, immunohistochemistry, etc.). In certain embodiments the control comprises a cell contacted with the test agent at a lower concentration or a cell or tissue not contacted with the test agent. In various embodiments the test agent is not an antibody, and/or not a protein, and/or not a nucleic acid. In certain embodiments the test agent is a small organic molecule.

In still another embodiment, this invention provides a method of prescreening for an agent that modulates an organism's response to a substance of abuse. The method typically involves contacting a component of a GDNF pathway or a BDNF pathway or a nucleic acid encoding the component with a test agent; and detecting specific binding of the test agent to the component or to the nucleic acid, where specific binding of the test agent to the component or to the nucleic acid indicates that the agent is likely to mitigate the organism's response to a substance of abuse. The method can optionally further involve recording test agents that specifically bind to the nucleic acid or to the component in a database of candidate agents that alter an organism's response to a substance of abuse. In certain embodiments the pathway component is not a D3 receptor. In certain embodiments the test agent is not an antibody and/or not a protein, and/or not a nucleic acid. In certain embodiments the test agent is a small organic molecule. In certain embodiments the detecting comprises detecting specific binding of the test agent to the nucleic acid (e.g., via a Northern blot, a Southern blot using DNA derived from an GDNF or BDNF pathway RNA, an array hybridization, an affinity chromatography, and an in situ hybridization, and the like). In certain embodiments the detecting comprises detecting specific binding of the test agent to the pathway component (e.g., via capillary electrophoresis, a Western blot, mass spectroscopy, ELISA, immunochromatography, and immunohistochemistry, and the like). In certain embodiments the test agent is contacted directly to the component or to the nucleic acid and/or to a cell containing the component, and/or the test agent is administered to an organism comprising the cell.

Also provided is a kit for mitigating one or more symptoms of substance abuse. The kit typically includes a container containing an agent that increases expression and/or activity of a component of a GDNF pathway and/or a BDNF pathway; and instructional materials teaching the use of the agent to modulate an organism's response to a substance of abuse or to withdrawal therefrom.

### DEFINITIONS

The term “substance of abuse” refers to a substance that is psychoactive and that induces tolerance and/or addiction. Substances of abuse include, but are not limited to stimulants (e.g. cocaine, amphetamines), opiates (e.g. morphine, heroin), cannabinoids (e.g. marijuana, hashish), nicotine, alcohol. Substances of abuse include, but are not limited to addictive drugs.

The term “gene product” refers to a molecule that is ultimately derived from a gene. The molecule can be a polypeptide encoded by the gene, an mRNA encoded by a gene, a cDNA reverse transcribed from the mRNA, and so forth.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “antibody”, as used herein, includes various forms of modified or altered antibodies, such as an intact immunoglobulin, an Fv fragment containing only the light and heavy chain variable regions, an Fv fragment linked by a disulfide bond (Brinkmann et al. (1993) Proc. Natl. Acad. Sci. USA, 90: 547-551), an Fab or (Fab)′2 fragment containing the variable regions and parts of the constant regions, a single-chain antibody and the like (Bird et al. (1988) Science 242: 424-426; Huston et al. (1988) Proc. Nat. Acad. Sci. USA 85: 5879-5883). The antibody may be of animal (especially mouse or rat) or human origin or may be chimeric (Morrison et al. (1984) Proc Nat. Acad. Sci. USA 81: 6851-6855) or humanized (Jones et al. (1986) Nature 321: 522-525, and published UK patent application #8707252).

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part 1, chapt 2, Overview of principles of hybridization and the strategy of nucleic acid probe assays, Elsevier, N.Y. (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, N.Y., and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

The phrase “expression or activity of a gene” (e.g., BNDF, GDNF gene, etc.) refers to the production of a gene product (e.g. the production of an mRNA and/or a protein) or to the activity of a gene product (i.e., the activity of a protein encoded by the gene).

The term “expression” refers to protein expression, e.g., mRNA and/or translation into protein. The term “activity” refers to the activity of a protein. Activities include but are not limited to phosphorylation, signaling activity, activation, catalytic activity, protein-protein interaction, transportation, etc. The expression and/or activity can increase, or decrease. Expression and/or activity can be activated directly or indirectly.

The “GDNF signaling pathway” refers to the pathway by which GDNF modulates an organism's response to ethanol or other substances of abuse. The GDNF signaling pathway includes, but is not limited to GDNF, GFRα1, RET, and the like.

A “GDNF expression pathway” refers to the pathway that regulates the level of GDNF available to activate the GDNF signaling pathway. It will be appreciated that agents that upregulate GDNF activity can do so by increasing expression or activity of the GDNF expression pathway (or one or more components thereof), and/or by increasing expression or activity of the GDNF signaling pathway (or one or more components thereof), or by agonizing GDNF activity, e.g., at the GDNF receptor (comprising RET). Thus, when referring, e.g., to a GDNF pathway and/or GDNF pathway component, for example in a screening assay, this reference is intended to include both the GDNF signaling pathway and the GDNF expression pathway.

A “GDNF pathway component” refers to a protein or other component comprising the pathway that regulates GDNF production or the pathway that mediates GDNF signaling. Thus, GDNF pathway components can include components that are “upstream” or “downstream” from GDNF in the pathway.

The “BDNF signaling pathway” refers to the pathway by which BDNF modulates an organism's response to ethanol or other substances of abuse (see, e.g., Yaka et al. (2003) J. Biol. Chem., 278(11): 9630-9638) and/or one or more components of the pathway by which BDNF modulates an organism's response to ethanol or other substances of abuse. The BNDF signaling pathway includes, but is not limited to BNDF, the dopamine D3 receptor (D3R), and the like.

A “BDNF expression pathway” refers to the pathway that regulates the level of BDNF available to activate the BDNF signaling pathway. It will be appreciated that agents that upregulate BDNF activity can do so by increasing expression or activity of the BDNF expression pathway (or one or more components thereof), and/or by increasing expression or activity of the BDNF signaling pathway (or one or more components thereof), or by agonizing BDNF activity, e.g., at the BDNF receptor (comprising TrkB). Thus, when referring, e.g., to a BDNF pathway and/or BGDNF pathway component, for example in a screening assay, this reference is intended to include both the BDNF signaling pathway and the BDNF expression pathway.

A “BDNF pathway component” refers to a protein or other component comprising the pathway that regulates BDNF production or the pathway that mediates BDNF signaling. Thus, BDNF pathway components can include components that are “upstream” or “downstream” from BDNF in the pathway.

### BRIEF DESCRIPTION OF THE DRAWINGS

FIG. 1, panels A through G show that ibogaine decreases ethanol consumption. Panel A: ibogaine decreased ethanol consumption during continuous access to both ethanol and water in the home cage when intake was measured 24 hours after an acute injection. Ethanol consumption expressed as grams of ethanol per kilogram body weight (g/kg+S.E.M.) was dose-dependently decreased by ibogaine administration as compared to vehicle administration [F(2,16)=5.12, P<0.02]. *P<0.05 as compared to vehicle treatment. (n=9). Panel B: ibogaine treatment decreased ethanol preference. Data are expressed as mls ethanol/(mls ethanol+mls water)±SEM. Ibogaine injection dose-dependently decreased ethanol preference as compared to vehicle treatment [F(2,16)=7.83, P<0.005]. *P<0.05 as compared to vehicle treatment. (n=9). Panel C: ibogaine did not affect sucrose preference. Data are expressed as mls sucrose/(mls sucrose+mls water)±SEM. Ibogaine did not alter sucrose preference measured 24 hours after injection, as compared to vehicle injection [F(1,8)=0.02, P=0.88]. (n=9). Panel D: ibogaine attenuated operant ethanol self-administration in rats. Systemic ibogaine injected 3 hr before an ethanol self-administration session reduced responding for oral ethanol at the active lever, but not the inactive lever [Main effect of Treatment: F(1,7)=5.77, P<0.05; Main Effect of Lever: F(1,7)=15.83, P<0.006; Treatment×Lever interaction: F(1,7)=5.78, P<0.05].*P<0.05 as compared to active lever responding following vehicle treatment. (n=8). Panel E: Subjects that supplied the results depicted in FIG. 1, panel D, were used to examine the effects of ibogaine on ethanol-induced relapse after a period of extinction training. Ibogaine injected 3 hours before the reinstatement test session reduced responding for ethanol at the active lever, but not the inactive lever [Main effect of Treatment: F(1,6)=11.16, P<0.02; Main effect of Lever F(1,6)=12.63, P<0.02; Treatment×Lever interaction F(1,6)=11.08, P<0.02]. Data are shown as mean±SEM.*P<0.02 as compared to active lever responding after vehicle injection. (n=8). Panel F: ibogaine does not induce a place preference or place aversion. DBA mice do not show preference or aversion to the ibogaine-paired chamber as indicated by a lack of a significant difference between subjects that received ibogaine in one chamber and saline in the other, and those that received only saline in both chambers when ibogaine treatment was given immediately [t=1.59, p=0.13] or 3 hrs [t=0.68, P=0.51] before training. Data are shown as time spent at test in the ibogaine (or saline)-paired chamber—time in the saline-paired chamber±SEM. (n=8 per group). Panel G: ibogaine does not alter acquisition of ethanol-induced CPP. Significant ethanol-induced CPP is observed following pretreatment with either vehicle or ibogaine 3 hours before the ethanol CPP training. There was no effect of ibogaine treatment [t=0.17, P=0.87]. Data are shown as time spent at test in the ethanol-paired chamber—time spend in the saline-paired chamber±SEM. (n=8 per group).

FIG. 2, panels A through D show that iIntra-VTA microinjection of ibogaine decreases ethanol self-administration and systemic ibogaine increases GDNF expression in a midbrain region that contains the VTA. Panels A-B: Intra-VTA ibogaine decreased ethanol self-administration by rats. Panel A: ibogaine microinjected into the VTA 3 hours before an ethanol self-administration session dosedependently decreased mean lever press responding relative to aCSF vehicle microinjection [F(3,27)=6.89, P<0.001]. (n=10). Panel B: Time course of effect of Intra-VTA ibogaine. The same subjects in (A) were tested 24 and 48 hours after ibogaine treatment. Mean responding on the day before treatment is shown as Baseline. There was no difference in baseline responding prior to treatment [F(3,27)=0.36, P=0.78]. Ibogaine micro-injection reduced lever responding for ethanol, and this reduction was long-lasting [Main effect of Concentration: F(3,54)=8.62, P<0.001, Main Effect of Time: F(2,54)=6.76, P<0.006, Concentration×Time interaction: F(6,54)=0.26, P=0.95]. The 10 μM dose significantly reduced responding at all time points as compared to vehicle treatment. The 0.1 μM dose was omitted from this graph for clarity; responding following this dose did not differ from responding following vehicle at any tested time point. Data are shown as mean±SEM. *P<0.05, **P<0.001 as compared to active lever responding after vehicle injection (n=11). Panels C and D: The midbrain region was excised 1, 6 or 12 h post i.p. injection of 40 mg/kg ibogaine. The expression of GDNF and control GPDH in mouse (panel C) and rat (panel D) were analyzed by RTPCR. Histogram depicts the mean ratio (GDNF/GPDH)±SD of 3 experiments (*P<0.05).

FIG. 3, panels A through G, shows that ibogaine activates the GDNF pathway in SHSY5Y cells. Panel A: Cells were treated with 10 μM ibogaine for the indicated times and lysed for total RNA isolation. Expression of GDNF and control Actin were analyzed by RT-PCR (n=4). Panel B: Cells were treated with 10 μM of ibogaine for the indicated times. GDNF in the media was detected by an ELISA assay. Histogram depicts the mean±SD of GDNF secretion in 3 experiments. Panel C: Cells were treated with the indicated concentrations of ibogaine for 3 hours. Ret was immunoprecipitated with anti-Ret antibodies followed by Western blot analysis with anti GFRα1 antibodies. The levels of GFRα1 in the homogenates were determined by Western blot analysis (n=3). Panel D: Cells were treated with 10 mM ibogaine for the indicated time point. Ret was immunoprecipitated followed by Western blot analysis with anti-phospho tyrosine or anti-Ret antibodies (n=3). Panel E: Cells were treated with vehicle (lane 1), or 10 μM ibogaine (lane 2) for 3 hours or with 50 ng/ml BDNF for 10 minutes (lane 3). Trk phosphorylation was analyzed by Western blot analysis with anti-phospho-Trk antibodies. The levels of TrkB were also determined by Western blot analysis (n=3). Panel F: Cells were pre-incubated with 0.3 unit/ml PI-PLC for 1 h (lanes 3 and 4). Cells were then washed and treated without (lanes 1 and 3) or with 10 μM ibogaine (lanes 2 and 4) for 3 hours. Ret phosphorylation was determined as described above (n=4). Panel G: Cells were treated with vehicle (lane 1), with 10 N^{o}M ibogaine alone (lane 2) or together with 10 N^{o}g/ml of mouse IgG (lane 3) or anti-GDNF neutralizing antibodies (lane 4) for 3 hours. Treatment with 50 ng/ml GDNF was used as a positive control (lane 5). The cells were lysed and Ret phosphorylation was analyzed as described above (n=3).

FIG. 4, panels A through C, shows that ibogaine activates the MAP kinase signaling pathway. Panel A: SHSY5Y cells were treated with 10 μM ibogaine for the indicated times. ERK2 and pERK1/2 were detected by Western blot analysis with anti-ERK2 and pERK1/2 antibodies respectively. Line-graph depicts the mean ratio (pERK2/ERK2)±SD of 3 experiments. Panel B: Cells were pre-incubated with the inhibitors U0126 (20 μM) (lanes 3 and 4) and PD58089 (40 μM) (lanes 5 and 6) for 30 minutes and then treated without (lanes 1, 3 and 5) or with 10 μM ibogaine (lanes 2, 4 and 6) for 3 hours (n=3). Panel C: Cells were pre-incubated with 0.3 unit/ml PI-PLC for 1 h (lane 3). Cells were then washed and treated without (lanes 1) or with 10 μM ibogaine (lanes 2 and 3) for 3 hours (n=3).

FIG. 5, panels A through C, shows that ibogaine modulates ethanol-induced changes in TH protein level via GDNF. Panel A: SHSY5Y cells were treated with different concentrations (0, 10, 25, and 100 mM) of ethanol for 24 hours. TH protein levels and control Actin levels were determined by Western blot analysis. Histogram depicts the mean ratio (TI/actin)±SD of 3 experiments. Panel B: Cells were treated with media (control), 100 ng/ml of GDNF for 12 h (lane 1), 100 mM ethanol for 24 hours (lane 2), or 100 mM ethanol for 24 hours to which 100 ng/ml GDNF was added for the last 12 hours (lane 3). The cells were lysed for Western blot analysis. Histogram depicts the mean ratio (TH/actin)±SD of 3 experiments. Panel C: Cells were treated with media (control), 10 N^{o}M ibogaine for 12 hours (lane 1), 100 mM ethanol for 24 hours to which 10 N^{o}M ibogaine was added for the last 12 h of ethanol incubation (lanes 3-5), after preincubation with PI-PLC (lane 4, see FIG. 3F), or together with 10 N^{o}g/ml of anti-GDNF neutralizing antibodies (lane 5). Histogram depicts the mean ratio (TH/actin)±SD of 3 experiments.

FIG. 6 shows that intra-VTA infusion of anti-GDNF neutralizing antibodies attenuates the effects of ibogaine on ethanol self-administration. Rats were implanted with bilateral osmotic minipumps for continuous delivery of anti-GDNF neutralizing antibodies or mouse IgG (600 ng/12 μl/side/day) into the VTA. After 2 days recovery, the rats were received 7 daily 1-hour ethanol self-administration sessions, followed by an injection of 40 mg/kg ibogaine (i.p.) on the 8th day. This figure depicts performance of subjects before and after ibogaine injection. The baseline data, Pre-Pump and Pre-Ibo, represent the average of the last three training sessions before pump connection or ibogaine injection, respectively. There was no effect of intra-VTA anti-GDNF antibody infusion on baseline levels of ethanol self-administration (Pre-Pump vs. Pre-Ibo) [F(1,15)=0.244, P=0.63]. When the Pre-Ibo baseline was compared with data obtained 3 and 24 hours after ibogaine injection, there was a significant effect of antibody treatment [F(1,30)=5.15, P<0.04] and a significant effect of time [F(2,30)=23.1, P<0.001]. Although ibogaine treatment decreased responding in both groups relative to the Pre-Ibo baseline, the decrease in responding is significantly greater in the control mouse IgG group than in subjects treated with anti-GDNF neutralizing antibodies. **P<0.02, *P<0.05. The data are shown as mean lever presses±SEM. (n=9 per group).

FIG. 7, panels A through D, shows that ethanol increases BDNF expression. Panels A-B: Ethanol exposure induces BDNF expression in primary dissociated hippocampal neurons. Three-week cultured neurons were treated with (panel A) 25 mM KCl (positive control), or the absence or presence of increasing concentrations of ethanol for 0.5 hours, or (panel B) with 100 mM ethanol for 0-48 hours. Following treatment, RNAs were isolated and expression of BDNF and control GPDH were analyzed using RT-PCR. Histogram depicts the mean ratio of BDNF/GPDH±SD, n=3. *P<0.05, **P<0.01 compared to control. Panels C-D: Increase of BDNF but not NGF expression in the striatum, but not the prefrontal cortex, of mice following ethanol self-administration. C57 mice were allowed continuous access to ethanol for 2 weeks using the two bottle (water and ethanol) choice procedure. The baseline intake the last 3 days prior to dissection was 9.66+0.55 g/kg/24 hrs (n=5, range 8.01-11.97). Brain regions were dissected 3 hours after the start of the dark cycle after a mean intake of 4.95±0.56 g/kg. BDNF, NGF and control GPDH were analyzed in punched (panel C) striatum or (panel D) prefrontal cortex tissue by RT-PCR. Histograms depicts the mean ratio of neurotrophin/GPDH±SD, n=4. *P<0.05 compared to mice consuming water only.

FIG. 8, panels A-D, shows that BDNF regulates the behavioral effects of ethanol. Panel A: Inhibition of the TrkB receptor increases ethanol intake in BDNF^{+/+} mice. Wildtype mice received intraperitoneal (i.p.) injections of K252a (black bar, 5 or 25 N^{o}g/kg) or vehicle (white bar). (n=21). *P<0.05, **P<0.01 compared to vehicle. Panel B: BDNF^{+/+} (black bar, n=17) mice have enhanced conditioned place preference (CPP) to ethanol (2 g/kg, i.p.), as compared to BDNF^{+/+} wildtype mice (white bar, n=21). The CPP score was calculated as [time spent in the ethanol-paired chamber at test]−[time spent in the ethanol-paired chamber during habituation]. *P<0.05. Panel C: BDNF^{±} mice have enhanced ethanol sensitization. The travel distance was measured every 5 minutes for 15 minutes after challenge with 2.0 g/kg ethanol in BDNF^{+/+} mice (n=14) and BDNF^{±} mice (n=10) on Day 5 (white circles) and Day 16 (black circles). **P<0.01 as compared to the same time point for the BDNF^{+/+} mice. Panel D: Ethanol consumption is enhanced in BDNF^{±} mice after a 2-week withdrawal period. BDNF^{±} (black circle, n=9) and BDNF^{+/+} (white circle, n=11) mice were allowed continuous access to 20% ethanol using the two-bottle choice procedure for 2 weeks, followed by a period of 2 weeks in which no ethanol was available. Intake was measured for 4 days after after 20% ethanol was reintroduced.

FIG. 9, panels A and B, shows that ethanol induces BDNF expression through RACK1 nuclear translocation in primary hippocampal neurons. Panel A: Ethanol induces RACK1 translocation into nucleus. Primary hippocampal neurons were exposed to 100 mM ethanol for 30 minutes and RACK1 nuclear localization was visualized by confocal microscopy by the merged signal (yellow-orange) between the anti-RACK1 antibody (green), and the nuclear marker TOTO-3 (red). Images are representative of approximately 10,000 cells in 5 experiments. Panel B: Transduction of Tat-tagged N-terminal fragment of RACK1 (Tat-RACK1ΔC) inhibits ethanol-induced BDNF expression in hippocampal neurons. Neurons were incubated with vehicle (control, lane 1), with 100 mM ethanol for 30 minutes (lane 2), with 1 μM Tat-RACK1ΔC for 60 minutes (lane 3), or pre-incubated with 1 μM TatRACK1ΔC for 30 minutes and then treated with 100 mM ethanol for an additional 30 minutes (lane 4). Histogram depicts the mean ratio of BDNF/GPDH±SD, n=3. *P<0.05 compared to ethanol alone. **P<0.01 compared to control.

FIG. 10, panels A-D, shows that tat-RACK1 induces expression and secretion of BDNF in dissociated hippocampal neurons. Panel A: Tat-RACK1 is transduced throughout the cell including the nucleus. Cells were treated with vehicle or 1 μM Tat-RACK1 for 2 hours. HA-tagged Tat-RACK1 nuclear localization was visualized by confocal microscopy by the merged signal (yellow-orange) between the anti-HA antibody for the detection of Tat-RACK1 (green), and the nuclear marker TOTO-3 (red). Images are representative of approximately 10,000 cells in 5 experiments. Panel B: Tat-RACK1 induces BDNF expression in primary dissociated hippocampal neurons. Neurons were treated with 1 N^{o}M Tat-RACK1 for the indicated times and BDNF and GPDH expression were analyzed by RT-PCR. Histogram depicts the mean ratio of BDNF/GPDH±SD, n=3. *P<0.05, **P<0.01 compared to control. Panels C-D: Tat-RACK1 increases the secretion of BDNF but not NGF in primary hippocampal neurons. Neurons were incubated without (control) or (panel C) with increasing concentrations of Tat-RACK1 for 6 hours, (panel D) or with 1 μM of Tat-RACK1, Tat-KIP²⁷, or the Tat-peptide for 6 hours, and BDNF (panels C-D) or NGF (panel D) secretion was determined. Histogram depicts the mean±SD from 3 experiments that were conducted in triplicate following treatment with vehicle (control, white bar), Tat-RACK1 (black bar), Tat-KIP²⁷ (gray bar), or Tat-peptide (hatched bar). **P<0.01 compared to control.

FIG. 11, panels A-F, shows that tat-RACK1 induces BDNF expression in the striatum. Panel A: Striatal slices were incubated with vehicle (white bar) or 1 μM Tat-RACK1 (black bars) for the indicated time. BDNF and GPDH expression were analyzed by RT-PCR. Histogram depicts the mean ratio of BDNF/GPDH±SD, n=3. *P<0.05, **P<0.01 compared to control. Panel B: Tat-RACK1 increases BDNF protein levels in striatal slices. BDNF and NGF protein levels in the striatum were measured following treatment with 1 μM Tat-RACK1 (white bars) or 1 μM Tat-KIP²⁷ (black bar) for 4 hours. Data are presented as the mean percent of control±SD (n=3). *P<0.05. Panel C: Tat-RACK1 increases the phosphorylation of the Trk receptor. Striatal slices were incubated with vehicle, 1 N^{o}M Tat-RACK1, or 1 N^{o}M Tat-RACK1 and 200 nM K252a for 4 hours. Slices were homogenized and protein extracts (50 μg) were analyzed by Western blot analysis. Membranes were probed with anti-phosphoTyr680/681Trk (top) and antiTrkB (bottom) antibodies. (n=3). Panel D: Tat-RACK1 is transduced into mouse brain after systemic injection. Mice were injected i.p. with vehicle or 4 mg/kg of Tat-RACK1. Four hours later, brains were immediately removed and homogenized and protein extracts were prepared. Samples (50 N^{o}g) were resolved on SDS-PAGE and membranes were probed with anti-RACK1 (top) or anti-HA (bottom) antibodies. Purified Tat-RACK1 (20 ng; Input) was also included. (E) Immunocytochemistry images show Tat-RACK1 is transduced into the striatum after i.p. injection. Mice were injected i.p. with vehicle or 4 mg/kg of Tat-RACK1 containing an HA tag. Saggital striatal sections were dissected 4 hours post-injection. Tat-RACK1 in the striatum was detected by using anti-HA antibodies. Scale bar, 50 μm. Panel F: Tat-RACK1 increases BDNF expression in vivo. Six hours following i.p. injection of vehicle or Tat-RACK1 (4 mg/kg) to mice, bilateral tissue punches of the striatum were homogenized for RNA isolation. BDNF, NGF, and GPDH expression were analyzed by RT-PCR. Top panel depicts results from 4 individual animals from each group. Histogram depicts the mean ratio of BDNF/GPDH±SD, n=4. **P<0.01 compared to vehicle.

FIG. 12, panels A-F, illustrates selective reduction in ethanol intake by Tat-RACK1. Panels A-B: I.P. injection to mice of 4 mg/kg (3-4 nmol per mouse) Tat-RACK1, but not TatKIP²⁷ (7.5 mg/kg, 8-9 nmol per mouse), reduced ethanol intake in the two-bottle choice procedure. Tat-RACK1 reduced ethanol intake as measured by (panel A) grams per kilogram body weight, and (panel B) milliliters of ethanol (black bars) and water (white bars) consumed. (panels A-B) Injection of Tat-KIP²⁷ did not affect ethanol or water intake. (n=11 for Tat-RACK1 and vehicle; n=4 for Tat-KIP²⁷). **P<0.005 compared to vehicle. Panel C: Tat-RACK1 decreased preference for ethanol (n=11) as well as for a sucrose (n=12) solution, but did not affect preference for a quinine solution (n=12). Preference score is calculated as [experimental solution (mls)/total fluid (mls)]*P<0.05, **P<0.005 compared to vehicle. Panels D-E: Intracerebroventricular (i.c.v.) administration of 1 N^{o}M (2 μg/5 μl ; 50 pmol) TatRACK1 reduces ethanol intake in rats. Rats were microinjected i.c.v. with vehicle (n=8, white bar), Tat-RACK1 (n=8, black bar) and Tat-peptide (n=5, gray bar). Ethanol consumption was measured by (panel D) grams per kilogram body weight, and (panel E) milliliters of ethanol (black bars) and water (white bars) consumed. *P<0.01 compared to vehicle. Panel F: Tat-RACK1 injection blocks the expression of ethanol behavioral sensitization. Three hours before the ethanol challenge on Day 16, mice received vehicle (white circles, n=11) or Tat-RACK1 (4 mg/kg) (black circles, n=11). Data are shown as total travel distance in 15 min after ethanol challenge injection. *P<0.01 compared to vehicle treatment.

FIG. 13, panels A-B, illustrates that tat-RACK1 reduces ethanol consumption via BDNF. Panel A: Tat-RACK1 effects are reduced in BDNF^{±} mice. Mice were exposed to ethanol using the two-bottle choice procedure. There was no effect of genotype on baseline ethanol intake (g/kg, mean±SEM: wild type, 9.22±0.64; heterozygote, 7.83±0.82). Data are percent change in grams per kilogram of ethanol consumed after vehicle injection (100×[Tat-RACK1_{_{g}}_{/k}_{_{g}}−Vehicle_{g/kg}]/Vehicle_{g/kg}) following i.p. injection of 4 mg/kg Tat-RACK1 to BDNF^{+/+} (white bars, n=17) and BDNF^{±} (black bars, n=11) mice. *P<0.05, compared to BDNF^{+/+} mice. Panel B: K252a inhibits the effect of Tat-RACK1 on ethanol consumption. Mice were exposed to ethanol using the two-bottle choice procedure. Effect of i.p. injection of vehicle (white bar), Tat-RACK1 (4 mg/kg) (black bar) or Tat-RACK1 (4 mg/kg) +K252a (25 ag/kg) (gray bar) on ethanol intake as measured by g/kg (n=12). There was no effect of any treatment on water consumption (data not shown). *P<0.05.

FIG. 14 illustrates that RACK1 and BDNF are part of a homeostatic pathway that regulates ethanol intake. (1) Acute ethanol exposure results in the translocation of RACK1 to the nucleus. Nuclear RACK1 mediates an increase in BDNF expression. (2) Increasing the protein levels of RACK1 via Tat-RACK1 transduction also results in elevated levels of BDNF. (3) An increase in BDNF expression leads to an increase in the secreted BDNF protein. (4) Secreted BDNF activates the BDNF pathway resulting in a reduction of ethanol's reinforcing/rewarding properties.

### DETAILED DESCRIPTION

In one embodiment, this invention pertains to the discovery that iobgaine reduces (mitigates) one or more symptoms associated with drug abuse (e.g., self administration, relapse, withdrawal, etc.) and that this effect is mediated by an increase in glial cell line-derived neurotrophic factor (GDNF). In particular, it was observed that ibogaine decreased ethanol intake by rats in 2-bottle choice and operant self-administration paradigms. Ibogaine also reduced operant selfadministration of ethanol in a reinstatement model. Using a conditioned place preference (CPP) paradigm, it was found that ibogaine was not rewarding or aversive, nor did it alter the rewarding properties of ethanol.

Microinjection of ibogaine into the ventral tegmental area (vta) reduced self-administration of ethanol, and systemic administration of ibogaine increased the expression of the glial cell line-derived neurotrophic factor (GDNF) in a midbrain region that includes the VTA. In dopaminergic neuron-like SHSY5Y cells, ibogaine treatment upregulated the GDNF pathway, and reversed ethanol's effect on tyrosiile hydroxylase (TH) protein levels via a GDNF-dependent mechanism. Finally, the ibogaine-mediated decrease in ethanol self-administration was reduced by intra-VTA delivery of anti-GDNF neutralizing antibodies. Together, these results indicate that GDNF in the VTA mediates the action of ibogaine on ethanol consumption. These findings highlight the importance of the GDNF or components of the GDNF pathway (e.g. GFRα1, RET, etc.) as news target for medications for alcoholism, withdrawal, or consumption of other substances of abuse that can mimic the effect of ibogaine against consumption of substances of abuse, while avoiding the negative side effects.

Thus, in one embodiment, this invention provides a method of identifying an agent that mitigates one or more symptoms of substance abuse, where the method involves contacting a cell or tissue with a test agent; and determining whether or not there is an increase in expression or activity of a GDNF pathway component (e.g. GDNF, GFRα1, RET, etc.), wherein an increase in expression or activity of a GDNF pathway or component, as compared to a control, indicates that the agent is an agent that mitigates a symptom of substance abuse.

In another embodiment, this invention exploits the GDNF pathway to mitigate one or more symptoms of substance abuse or withdrawal therefrom. Thus, in certain embodiments, this invention also provides a method of mitigating one or more symptoms (e.g., self administration, preference, relapse, withdrawal) of substance abuse in a mammal, where the method involves increasing the level, expression, or activity of GDNF (and/or the GDNF pathway) in the mammal.

In another embodiment, this invention pertains to the discovery that brain-derived neurotrophic factor (BDNF) and its associated signaling pathway is involved in reversing and/or counteracting neuroadaptations within the mesolimbic system that contribute to the development and/or maintenance of addiction (e.g., alcohol addiction). Moreover, it is demonstrated herein that inhibition of the BNDF signaling pathway (e.g. by administration of a dominant negative fragment of RACK1 (RACK1ΔC) blocked the ethanol-induced elevation of BDNF expression. It was also demonstrated that upregulation of the BDNF pathway, e.g. by in vivo administration of RACK1 (tat-RACK1 fusion protein) reduced self-administration of ethanol and that this reduction of ethanol consumption was mediated by increasing levels of BDNF.

These discoveries can be exploited to treat substance abuse (e.g. mitigate one or more symptoms of substance abuse and/or withdrawal from chromic consumption of a substance of abuse) and/or to screen for agents useful for mitigating one or more symptoms of substance abuse and/or withdrawal. Thus, in certain embodiments, this invention provides a method of mitigating one or more symptoms of substance abuse in a mammal. The method typically involves increasing the expression or activity of BDNF, RACK1, and/or the dopamine D3 receptor (D3R) in the mammal. Also provided is a method of identifying an agent that mitigates one or more symptoms of substance abuse. The method typically involves contacting a cell or tissue with a test agent; and determining whether or not there is an increase in increase in expression or activity of a component of a BDNF pathway, wherein an increase in expression or activity of a component of a BDNF pathway, as compared to a control, indicates that the test agent is an agent that mitigates a symptom of substance abuse.

I. Assays for Agents that Modulate Expression or Activity of GDNF or One or More Components of the BDNF Signaling Pathway.

As indicated above, in one aspect, this invention pertains to the discovery that the BDNF pathway is involved in reversing and/or counteracting neuroadaptations within the mesolimbic system that contribute to the development and/or maintenance of addiction to substances of abuse (e.g. cocaine, alcohol, nicotine, cannabis, etc.). In addition, this invention pertains to the discovery that the effects of ibogaine are mediated by GDNF (e.g., in the VTA). Thus, agents that increase expression or activity of the BDNF pathway or components thereof and/or agents that increase the expression and/or activity of the GDNF pathway attenuate one or more symptoms of substance abuse and are expected to have prophylactic and/or therapeutic utility as described herein. Thus, in certain embodiments this invention provides methods of screening for agents that modulate (e.g., increase) the activity and/or expression of one or more components of the BDNF pathway and/or one or more components of the GDNF pathway.

The methods typically involve detecting alteration(s) of expression and/or activity of the protein(s) of interest (e.g., BDNF, GDNF, etc.) in response to administration of one or more test agent(s) to a cell, tissue, or animal. In certain embodiments, an elevated expression level or activity level produced by the agent as, e.g., compared to a negative control where the test agent is absent or at reduced concentration indicates that the agent upregulates activity or expression of the factor(s) in question. Conversely, decreased expression level, or activity level, resulting from treatment by the agent as compared to a negative control where the test agent is absent or at reduced concentration indicates that the agent down-regulates expression or activity of the factor(s).

A) Assaying for Modulators of BDNF Pathway Component Expression or GDNF Pathway Component Expression.

Expression levels of a gene can be altered by changes in by changes in the transcription of the gene product (i.e., transcription of mRNA), and/or by changes in translation of the gene product (i.e., translation of the protein), and/or by post-translational modification(s) (e.g. protein folding, glycosylation, etc.). Thus preferred assays of this invention typically contacting a test cell, tissue, or animal with one or more test agents, and assaying for level of transcribed mRNA (or other nucleic acids derived from the neurotrophic and/or neurogenerative factor gene(s)), level of translated protein, activity of translated protein, etc. Examples of such approaches are described below.

It will be noted that where the description below refers simply to GDNF, it applies equally to assaying for modulators of expression or activity of any one or more components of a GDNF pathway (e.g., GDNF, GFRα1, RET, etc.) and/or any one or more components of a BDNF (BDNF) pathway (e.g. BDNF, RACK1, D3R, etc.).

1) Nucleic-Acid Based Assays.

a. Target Molecules.

Changes in expression level can be detected by measuring changes in mRNA and/or a nucleic acid derived from the mRNA (e.g. reverse-transcribed cDNA, etc.). In order to measure GDNF expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N. Y. and Tijssen ed.

In a preferred embodiment, the “total” nucleic acid is isolated from a given sample using, for example, an acid guanidinium-phenol-chloroform extraction method and polyA+mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g, Innis, et al., (1990) PCR Protocols. A guide to Methods and Application. Academic Press, Inc. San Diego,), ligase chain reaction (LCR) (see Wu and Wallace (1989) Genoinics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of factor(s) of interest in a sample, the nucleic acid sample is one in which the concentration of the GDNF pathway component or BDNF pathway component) or the concentration of the nucleic acids derived from the GDNF pathway component and/or BNDF pathway component, and/or mRNA transcript(s) encoding one or more of these components, is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript, or large differences or changes in nucleic acid concentration are desired, no elaborate control or calibration is required.

In the simplest embodiment, the nucleic acid sample is the total mRNA or a total cDNA isolated and/or otherwise derived from a biological sample (e.g., a sample from a neural cell or tissue). The nucleic acid may be isolated from the sample according to any of a number of methods well known to those of skill in the art as indicated above.

b. Hybridization-Based Assays.

Using the known sequence of various GDNF pathway components and/or BDNF pathway components, detecting and/or quantifying the transcript(s) can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed GDNF mRNA), typically fragmented and separated on an electrophoretic gel, is hybridized to a probe specific for the nucleic acid encoding the GDNF. Comparison of the intensity of the hybridization signal from the target specific probe with a “control” probe (e.g. a probe for a “housekeeping gene) provides an estimate of the relative expression level of the target nucleic acid.

Alternatively, the GDNF pathway component and/or BDNF pathway component RNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes can be used to identify and/or quantify the target mRNA. Appropriate controls (e.g. probes to housekeeping genes) can provide a reference for evaluating relative expression level.

An alternative means for determining the GDNF pathway component and/or BDNF pathway component expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use can vary depending on the particular application.

In some applications it is necessary to block the hybridization capacity of repetitive sequences. Thus, in some embodiments, tRNA, human genomic DNA, or Cot-1 DNA is used to block non-specific hybridization.

c. Amplification-Based Assays.

In another embodiment, amplification-based assays can be used to measure GDNF pathway component and/or BDNF pathway component expression (transcription) level. In such amplification-based assays, the target nucleic acid sequences (e.g., GDNF nucleic acid(s)) act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate (e.g. healthy tissue or cells unexposed to the test agent) controls provides a measure of the transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One suitable internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The known nucleic acid sequence(s) for GDNF are sufficient to enable one of skill to routinely select primers to amplify any portion of the gene.

d. Hybridization Formats and Optimization of Hybridization

i. Array-Based Hybridization Formats.

In certain embodiments, the methods of this invention can be utilized in array-based hybridization formats. Arrays typically comprise a multiplicity of different “probe” or “target” nucleic acids (or other compounds) attached to one or more surfaces (e.g., solid, membrane, or gel). In certain embodiments, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

The simple spotting, approach has been automated to produce high density spotted arrays (see, e.g., U.S. Pat. No: 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

ii. Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C., or ³²P-labelled probes or the like. Other labels include ligands that bind to labeled antibodies, fluorophores, chemi-luminescent agents, enzymes, and antibodies which can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

e. Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25× SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results, and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

f. Labeling and Detection of Nucleic Acids.

The probes used herein for detection of GDNF pathway component and/or BDNF pathway component expression levels can be full length or less than the full length of the mRNA(s) encoding the particular component(s) of interest. Shorter probes are empirically tested for specificity. Preferred probes are sufficiently long so as to specifically hybridize with the target nucleic acid(s) under stringent conditions. The preferred size range is from about 20 bases to the full length of the encoding mRNA, more preferably from about 30 bases to the length of the mRNA, and most preferably from about 40 bases to the length of mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Detectable signal can also be provided by chemiluminescent and bioluminescent sources. Chemniluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

2) Polypeptide-Based Assays.

The GDNF pathway component and/or BDNF pathway component polypeptide(s) (e.g., GDNF, BDNF, RACK1, GFRα1, etc.) can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the GDNF pathway component and/or BDNF pathway component polypeptide(s) are detected/quantified in an electrophoretic protein separation (e.g., a 1- or 2-dimensional electrophoresis). Means of detecting proteins using electrophoretic techniques are well known to those of skill in the art (see generally, R. Scopes (1982) Protein Purification, Springer-Verlag, N. Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and can be directly labeled or alternatively may be subsequently detected using labeled antibodies (e.g., labeled sheep anti-mouse antibodies) that specifically bind to the a domain of the antibody.

In preferred embodiments, the GDNF pathway component and/or BDNF pathway component polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (e.g., GDNF proteins). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (e.g. GDNF protein) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind, e.g., GDNF, either alone or in combination. In the case where the antibody that binds the GDNF polypeptide(s) is not labeled, a different detectable marker, for example, an enzyme-labeled antibody capable of binding to the monoclonal antibody which binds the GDNF polypeptide, can be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention can also include other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds the GDNF polypeptide is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents can also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced using standard methods well know to those of skill in the art.

It will also be recognized that antibodies can be prepared by any of a number of commercial services (e.g., Berkeley antibody laboratories, Bethyl Laboratories, Anawa, Eurogenetec, etc.).

3) Reporter Based Assays.

In certain embodiments, the assays described herein utilize reporters to evaluate changes in GDNF and/or BDNF pathway activity. The reporters typically compirse reporter genes operably linked to promoters regulating transcription of one or more component in the BDNF and/or GDNF pathway. When the pathway is upregulated, the reporter gene expression is thereby increased and readily detected. Suitable reporter genes include, but are not limited to chloramphenicol acetyl transferase (CAT), luciferase, β-galactosidase (β-gal), alkaline phosphatase, horse radish peroxidase (HRP), and green fluorescent protein (GFP), red fluorescent protein (RFP), and the like.

4) Assays for Component Activity.

In certain embodiments, the test agent(s) can be evaluated for their ability to alter (e.g., increase) the activity of a GDNF pathway component and/or a BDNF pathway component. Such activity of a test agent might be via agonistic activity, e.g., at the GDNF receptor (RET) or at the BDNF receptor (TrkB). Thus for example, the test agent can be evaluated for its ability to increase the phorphorylated RET and/or TrkB (e.g., increase the ratio of phorphorylatred RET to unphosphorylated RET and/or ratio of phorphorylatred TrkB to unphosphorylated TrkB).

The level of phorphorylation of a protein can be determined by a variety of methods well known to those of skill in the art. For example, radioactively labeled phosphate may be added to cultured cells grown in both the presence and absence of the test agent(s). Proteins (e.g. RET) from the labeled cells can then be extracted and separated on a one or two dimensional gel system. Isolated phosphorylated proteins can then be visualized by autoradiography and related techniques. After separation and visualization, changes in the level of phosphorylation of different proteins may be determined by comparing the results obtained from cells exposed to the test agent(s) with the results obtained from cells not exposed to the test agent(s).

A more sensitive detection method involves the use of phosphoantibodies, for example, antibodies that recognize phosphorylated forms of specific proteins, or antibodies that recognize a phosphorylated amino acid residue, such as phosphothreonine or phosphoserine antibodies. Another useful detection method is back-phosphorylation, which is safer than direct phosphorylation assays but less sensitive. Cell extracts are incubated with radiolabeled ATP and Mg++ and subjected to gel electrophoresis. Where the test agent(s) alter protein phosphorylation a different amount of radiolabeled phosphate will be incorporated into individual proteins (e.g., RET) of cells exposed to the test agent(s) than in cells which have not been so exposed, resulting in a different pattern of bands on a gel.

5) Assay Optimization.

The assays of this invention have immediate utility in screening for agents that modulate the expression or activity a GDNF pathway component and/or BDNF pathway component by a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription the GDNF gene(s), nucleic acid based assays are preferred.

Routine selection and optimization of assay formats is well known to those of ordinary skill in the art.

II. Pre-Screening for Agents that Bind One or More Components of the GDNF Pathway and/or One or More Components of the BDNF Pathway.

In certain embodiments it is desired to pre-screen test agents for the ability to interact with (e.g. specifically bind to) a GDNF pathway component and/or a BDNF pathway component or to a nucleic acid encoding a component of the BDNF and/or the GDNF pathway. Specifically binding test agents are more likely to interact with the GDNF pathway and/or the BDNF pathway and thereby modulate GDNF expression and/or activity or expression or activity of the BDNF pathway. Thus, in some preferred embodiments, the test agent(s) are pre-screened for binding to a GNDF pathway component and/or to a component of the BDNF pathway or a nucleic acid encoding such, before performing the more complex assays described above.

It will be noted that where the description below is with respect to GDNF, it applies equally to assaying for modulators of expression or activity of components of the GDNF pathway and/or the BDNF pathway (e.g. GFRα1, RET, BDNF, RACK1, D3R, etc.).

In one embodiment, such pre-screening is accomplished with simple binding assays. Means of assaying for specific binding or the binding affinity of a particular ligand for a nucleic acid or for a protein are well known to those of skill in the art. In preferred binding assays, the GDNF pathway component and/or BDNF pathway component or the nucleic acid encoding GDNF pathway component and/or BDNF pathway component is immobilized and exposed to a test agent (which can be labeled), or alternatively, the test agent(s) are immobilized and exposed to GDNF pathway component and/or BDNF pathway component or to a nucleic acid encoding GDNF pathway component and/or BDNF pathway component (which can be labeled). The immobilized moiety is then washed to remove any unbound material and the bound test agent or bound protein or nucleic acid is detected (e.g. by detection of a label attached to the bound molecule). The amount of immobilized label is proportional to the degree of binding between the test agent and the GDNF pathway component and/or BDNF pathway component or to a nucleic acid encoding GDNF pathway component and/or BDNF pathway component.

III. Scoring the Assays.

As indicated above, methods of screening for modulators of expression or activity of component(s) of the GDNF pathway and/or the BDNF pathway typically involve contacting a cell, tissue, organism, or animal with one or more test agents and evaluating changes in nucleic acid transcription and/or translation or protein expression or activity. To screen for potential modulators, the assays described above are performed in the after administering and/or in the presence of one or more test agents using biological samples from cells and/or tissues and/or organs and/or organisms exposed to one or more test agents. The activity and/or expression level and/or interaction or the protein(s) of interest is determined and, in a preferred embodiment, compared to the activity level(s) observed in “control” assays (e.g., the same assays lacking the test agent). A difference between the nucleic acid or protein expression and/or activity and/or interaction in the “test” assay as compared to the control assay indicates that the test agent is a “modulator” of expression and/or activity and/or interaction of the desired protein(s).

In a preferred embodiment, the assays of this invention level are deemed to show a positive result, e.g. elevated expression and/or activity and/or interaction of GDNF, BDNF, etc. when the measured protein or nucleic acid level or protein activity is greater than the level measured or known for a control sample (e.g. either a level known or measured for a normal healthy cell, tissue or organism mammal of the same species not exposed to the or putative modulator (test agent), or a “baseline/reference” level determined at a different tissue and/or a different time for the same individual). In a particularly preferred embodiment, the assay is deemed to show a positive result when the difference between sample and “control” is statistically significant (e.g. at the 85% or greater, preferably at the 90% or greater, more preferably at the 95% or greater and most preferably at the 98% or greater confidence level).

IV. High Throughput Screening.

The assays of this invention are also amenable to “high-throughput” modalities. Conventionally, new chemical entities with useful properties (e.g., modulation of expression and/or activity of a component of a GDNF expression pathway and/or a GDNF signaling pathway, and/or BDNF expression pathway, and/or a BDNF signaling pathway are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A) Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries.

Any of the assays for agents that modulate expression and/or activity of component(s) of the GDNF and/or the BDNF pathway are amenable to high throughput screening. As described above, having determined that these components/pathways are associated with the molecular mechanisms underlying addiction, it is believe that modulators can have significant therapeutic value. Certain preferred assays detect increases of transcription (i.e., increases of mRNA production) by the test compound(s), increases of protein expression by the test compound(s), or binding to the gene (e.g., gDNA, or cDNA) or gene product (e.g., mRNA or expressed protein) by the test compound(s).

High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

V. Kits.

In still another embodiment, this invention provides kits for practice of the assays or use of the compositions described herein. In one preferred embodiment, the kits comprise one or more containers containing antibodies and/or nucleic acid probes and/or substrates suitable for detection of components of the GDNF and/or the BDNF pathway and/or activity levels. The kits can optionally include any reagents and/or apparatus to facilitate practice of the assays described herein. Such reagents include, but are not limited to buffers, labels, labeled antibodies, labeled nucleic acids, filter sets for visualization of fluorescent labels, blotting membranes, and the like.

In another embodiment, the kits can comprise a container containing a GDNF and/or a BDNF pathway protein, and/or a vector such a protein, and/or a cell comprising such a vector.

In certain embodiments, the kits comprise one or more agents (e.g., tat-RACK1) that increase activity of a GDNF pathway and/or a BDNF pathway.

In addition, the kits can optionally include instructional materials containing directions (i.e., protocols) for the practice of the therapeutic and/or assay methods of this invention or the administration of the compositions described here along with counterindications. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

VI. Modulator Databases.

In certain embodiments, the agents that score positively in the assays described herein (e.g. show an ability to increase the expression and/or activity, of a GDNF and/or BDNF pathway component) can be entered into a database of putative modulators of an organism's (e.g., a mammal's ) response to a substance of abuse. The term database refers to a means for recording and retrieving information. In certain embodiments the database also provides means for sorting and/or searching the stored information. The database can comprise any convenient media including, but not limited to, paper systems, card systems, mechanical systems, electronic systems, optical systems, magnetic systems or combinations thereof. Typical databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

VII. Altering Expression and/or Activity of a Component of a GDNF and/or a BDNF Pathway.

Expression and/or activity of the GDNF and/or the BDNF pathways can be upregulated by any of a variety of methods. Such methods include, but are not limited to the administration of one or more agents (e.g., ibogaine and ibogaine analogues) that upregulate activity of the pathway, introducing constructs expressing one or more components of the pathway (e.g. RACK1) into the target cell or tissue (e.g. using gene therapy approaches), upregulating endogenous expression of component(s) of the pathway(s) (e.g. using agents identified in the screening assays of this invention), and the like.

It is noted that ibogaine analogues are know those of skill in the art. Thus for example, two compounds that are structurally very similar to ibogaine, are ibogamine and tabemanthin. Like ibogaine, both of these are alkaloids isolated from Apocynaceae plants, (see, e.g., U.S. Pat. Nos: 5,152,994 and 5,026,697). Ibogamine is the basic five-ringed structure; ibogaine is 12-methoxyibogamine (ibogamine with a methoxy group added to the number 12 carbon atom), while tabernanthin is 13-methoxyibogamine (ibogamine with a methoxy group on the #13 carbon atom).

In certain embodiments, expression and/or activity of the GDNF and/or the BDNF pathways can be activated by the use of various small organic molecules (e.g. molecules identified according to the screening methods described herein. Such molecules include, but are not limited to molecules that specifically bind to the DNA comprising one or more components of the BDNF and/or GDNF pathway and/or to the component(s) themselves.

The mode of administration of the agent(s) to modulate GDNF and/or BDNF pathway activity depends on the nature of the particular agent. Antisense molecules, catalytic RNAs (ribozymes), catalytic DNAs, small organic molecules, and other molecules (e.g. lipids, antibodies, etc.) used as modulators of GDNF and/or BDNF pathway activity can be formulated as pharmaceuticals (e.g. with suitable excipient) and delivered using standard pharmaceutical formulation and delivery methods as described below. Antisense molecules, catalytic RNAs (ribozymes), catalytic DNAs, and additionally, knockout constructs, and constructs encoding intrabodies can be delivered and (if necessary) expressed in target cells (e.g. vascular endothelial cells) using methods of gene therapy that are well known to those of skill in the art (see, e.g., U.S. Pat. Nos. 6,743,620, 6,784,162, 6,645,942 and the references therein).

In certain embodiments, one or more activators of the GDNF and/or the BDNF pathway are administered to an individual to alter the response to ethanol or other substances of abuse, and/or to mitigate one or more symptoms/behaviors associated with consumption of a substance of abuse or withdrawal therefrom. While this invention is described generally with reference to human subjects, veterinary applications are contemplated within the scope of this invention.

Various activators may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

The active agents and various derivatives and/or formulations thereof are useful for parenteral, topical, oral, or local administration, such as by aerosol or transdermally, for prophylactic and/or therapeutic treatment of coronary disease and/or rheumatoid arthritis. The pharmaceutical compositions can be administered in a variety of unit dosage forms depending upon the method of administration. Suitable unit dosage forms, include, but are not limited to powders, tablets, pills, capsules, lozenges, suppositories, etc.

The active agent(s) and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

The concentration of active agent(s) in the formulation can vary widely, and will be selected primarily based on fluid volumes, viscosities, body weight and the like in accordance with the particular mode of administration selected and the patient's needs.

In therapeutic applications, the compositions of this invention are administered to a patient suffering from substance abuse (e.g. alcoholism) or withdrawal from chronic consumption of a substance of abuse) in an amount sufficient to eliminate or at least partially arrest one or more symptoms/behaviors associated with the condition. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) of the patient.

In certain preferred embodiments, the GDNF and/or the BDNF pathway modulators are administered orally (e.g. via a tablet) or as an injectable in accordance with standard methods well known to those of skill in the art. In other embodiments, GDNF and/or the BDNF pathway modulators can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

The foregoing formulations and administration methods are intended to be illustrative and not limiting. It will be appreciated that, using the teaching provided herein, other suitable formulations and modes of administration can be readily devised.

### EXAMPLES

The following examples are offered to illustrate, but not to limit the claimed invention.

### Example 1

### The Glial Cell Line-Derived Neurotrophic Factor Mediates the Desirable Actions of the Anti-Addiction Drug ibogaine against Alcohol Consumption

Alcohol addiction manifests as uncontrolled drinking despite negative consequences. Few medications are available to treat the disorder. Anecdotal reports suggest that ibogaine, a natural alkaloid, reverses behaviors associated with addiction including alcoholism; however, due to side effects, ibogaine is not clinically used. In this study we first characterized ibogaine's actions on ethanol self-administration in rodents. Ibogaine decreased ethanol intake by rats in 2-bottle choice and operant self-administration paradigms. Ibogaine also reduced operant selfadministration of ethanol in a reinstatement model. Using a conditioned place preference (CPP) paradigm, we found that ibogaine was not rewarding or aversive, nor did it alter the rewarding properties of ethanol. Next, we set to identify a molecular mechanism that mediates the desirable activities of ibogaine on ethanol intake. Microinjection of ibogaine into the ventral tegmental area (VTA) reduced self-administration of ethanol, and systemic administration of ibogaine increased the expression of the glial cell line-derived neurotrophic factor (GDNF) in a midbrain region that includes the VTA. In dopaminergic neuron-like SHSY5Y cells, ibogaine treatment upregulated the GDNF pathway, and reversed ethanol's effect on tyrosine hydroxylase (TH) protein levels via a GDNF-dependent mechanism. Finally, the ibogaine-mediated decrease in ethanol self-administration was reduced by intra-vta delivery of anti-GDNF neutralizing antibodies. Together, these results suggest that GDNF in the VTA mediates the action of ibogaine on ethanol consumption. These findings highlight the importance of the GDNF as a new target for medications for alcoholism that may mimic the effect of ibogaine against alcohol consumption, while avoiding the negative side effects.

Methods

Materials

ibogaine-HCl, phosphatidylinositol phospholipase C (PI-PLC), and anti-tyrosine hydroxylase (TH) antibodies were purchased from Sigma (St. Louise, Mo.). Human glial-cell line derived neurotrophic factor (GDNF) was purchased from Upstate Cell Signaling Solutions (Charlottesville, Va.). Anti-GDNF monoclonal neutralizing antibodies were purchased from R&D Systems (Minneapolis, Minn.). The inhibitors U0126 and PD98059 were purchased from Calbiochem (San Diego, Calif.). Protease inhibitor cocktail was purchased from Roche Applied Science (Indianapolis, Ind.). Anti-phospho-tyrosine antibodies were purchased from BD Transduction Laboratories (San Diego, Calif.). Anti-Ret, anti-GFRα1, anti-extracellular signalregulated kinase 2 (ERK2) and anti-phospho ERK1/2, anti-TrkB, anti-phosphoTrk antibodies and mouse IgG were purchased from Santa Cruz Biotechnology (Santa Cruz, Calif.). Trizol reagent was purchased from Invitrogen (Carlsbad, Calif.). GDNF Emax Immunoassay System, the Reverse Transcription System and PCR Master Mix were purchased from Promega Corporation (Madison, Wis.). Primers for PCR were synthesized by BioSource International (Camarillo, Calif.). The artificial cerebrospinal fluid was purchased from CMA Microdialysis (North Chelmsford, Mass.)

Animals

Adult Male Long Evans rats were purchased from Harlan Sprague Dawley (Indianapolis, Ind.), C57/B16 mice and DBA mice were purchased from Jackson Laboratories (Bar Harbor, Me.). Rodents were housed in ventilated polycarbonate cages with food and water available ad lib except as noted below, on a standard 12:12 light:dark schedule with lights on at 6 a.m. Subjects were group housed for all studies, except for the ethanol 2-bottle and operant self-administration studies, for which they were singly housed. All studies were conducted with approval by the Gallo Center Institutional Animal Care and Use Committee and were in accordance with “PHS Policy on Humane Care and Use of Laboratory Animals”, Office of Laboratory Animal Welfare, National Institutes of Health, revised 2002.

Cell Culture

SHSY5Y human neuroblastoma cells were grown in Dulbecco's modified Eagle's medium (DMEM) containing 10% fetal bovine serum (FBS) plus 100 units/ml penicillin and 100 μg/ml streptomycin. Prior to experiments, the medium was replaced with DMEM containing 1% FBS.

Reverse Transcription Polymerase Chain Reaction (RT-PCR)

Total RNA was isolated using Trizol reagent and reverse transcribed using Reverse Transcription System kit with the oligo (dT)15 primer at 42ûC for 30 minutes. GDNF, Actin or GPDH expressions were analyzed by RT-PCR. The GDNF primers were based on the coding frame of the human GDNF gene: upstream, 5′-TGC CAG AGG ATT ATC CTG ATC AGT TCG ATG3′ (SEQ ID NO: 1); and downstream, 5′-TTG TCG TAC GTT GTC TCA GCT GCA TCG CAA-3′ (SEQ ID NO:2). The Actin primers were based on the human Actin gene: upstream, 5′-TCA TGA AGT GTG ACG TTG ACA TC-3′ (SEQ ID NO:3); and downstream, 5′-AGA AGC ATT TGC GGT GGA CGA TG-3′ (SEQ ID NO:4). The GPDH primers were based on the rat GPDH gene: upstream, 5′-TGA AGG TCG GTG TCA ACG GAT TTG GC-3′ (SEQ ID NO:5); and downstream, 5′-CAT GTA GGC CAT GAG GTC CAC CAC-3′ (SEQ ID NO:6). PCR conditions were optimized to specifically ensure that the amplification reactions are within the linear range by testing a range of 25-40 PCR cycles. The optimal numbers of cycles used were: 30 cycles for controls GPDH and actin and 35 cycles for GDNF.

After completion of PCR, 10 μl of each product was separated by 1.8% agarose gel in Tris/acetic acid/EDTA buffer with 0.25N^{o}g/ml ethidium bromide, photographed by Eagle Eye II (Stratagene), and quantified by NIH Image 1.61.

Immunoprecipitation

Cells were collected and lysed in RIPA buffer (50 mM Tris-Cl, pH 7.6, 150 mM NaCl, 1 % NP40, 0.1% SDS, 0.5% sodium deoxycholate, 2 mM EDTA, protease inhibitor cocktail and 10 mM sodium orthovanadate). Homogenates were incubated with 5 N^{o}g anti-Ret antibody in TBS-T buffer (20 mM Tris-Cl, pH7.6, 137 mM NaCl and 1% NP-40) overnight at 4° C., followed by 2 hours incubation with Protein G agarose. Samples were separated on an SDS-PAGE gel for Western blot analysis.

ELISA

GDNF secretion in the medium was detected using GDNF Emax Immunoassay System according to (Balkowiec and Katz, 2000). GDNF concentration was interpolated from the standard curves (linear range of 15.6-1000 pg/ml).

Ibogaine Preparation and Treatment for in vivo Studies in Mice and Rats

ibogaine-HCl was dissolved in water to create a stock solution. The stock solution was further diluted by appropriate vehicle solution (saline or artificial cerebrospinal fluid (aCSF)) as needed. Injections were intraperitoneal (i.p.) and were given in injection volumes of 2 ml/kg for rats and 1 ml/100 g for mice.

Iboiaine Effects on Gene Expression in vivo

Male C57B16 mice or Long Evans rats were habituated with 3 saline injections over 3 days followed by i.p. administration of 40 mg/kg ibogaine-HCl. The animals were sacrificed 1, 6 or 12 hours post injection, and brain regions were excised and stored at −80° C. until use for analysis of GDNF expression by RT-PCR.

Ethanol and Sucrose Self-Administration in the Two-Bottle Preference Test

Male Long Evans rats were allowed continuous access to two bottles, one containing 10% ethanol (10 E) in tap water and the other containing tap water. Two months later, 20 or 40 mg/kg ibogaine-HCl or vehicle were injected i.p. and ethanol and water intake were measured 24 hours later. Each subject received each dose, with one injection per week. Next, subjects were exposed to 2 bottles, one containing 10% sucrose (10 S) in tap water and the other containing tap water. 4 days later, the effects of ibogaine were determined as above.

Ethanol Operant Self-Administration Training

The self-administration chamber contained two levers: an active lever for which presses resulted in delivery of 0.1 ml fluid reward, and an inactive lever for which responses were counted but no programmed events occurred. Rats were water restricted, and then trained to lever press for a 10 S/10 E reinforcer solution in 3 overnight sessions. On the 4th day, rats were allowed ad libitum access to water in the home cage, and were transferred to 1-hour daily sessions wherein responses were reinforced with 5 S/10 E on a fixed-ratio 3 (FR3) schedule. Sucrose was phased out of the reinforcer solution over the next 7 days until the solution consisted of 10 E only. Experimental manipulations began after 30 sessions when stable 10 E intake of >0.4 g/kg ethanol was attained. Ibogaine (40 mg/kg, i.p.) or vehicle was administered 3 hours prior to the start of an ethanol self-administration session. The average intake by the group receiving i.p. ibogaine was 0.45±0.13 g/kg. Intakes at these levels have been found to produce measurable levels in blood (Weiss et al., 1993[*______]) and brain (Ferraro et al. (1991) Alcoholism: Clin. Exp. Res. 15: 504-507).

Effects of Systemic Iboiaine Administration on Ethanol Reinstatement

The eight rats used in the operant self-administration study were also used to study ethanolseeking after a period of extinction. Following a period of stable responding for ethanol, rats underwent extinction in which no ethanol was delivered following active lever responses. After about 12-15 extinction sessions, most rats met the extinction criteria (<5 presses per session). The rats were then injected with ibogaine (40 mg/kg, i.p.), or vehicle, 3 hours before the reinstatement test session in which lever presses for the ethanol-associated lever resulted in the delivery of 0.1 ml 10 E (FR1 schedule).

Ventral Tegmental Area (VTA) Cannulation and Microiniection of Iboiaine

After ethanol self-administration was established as described above, subjects attained a mean daily intake of ethanol of 0.62±0.07 g/kg. Bilateral guide cannula (26 gauge, Plastics One) were implanted into the VTA (5.6 mm posterior to bregma, 1.0 mm mediolateral, 8.0 mm ventral to the skull surface). After 7 to 10 days recovery, sham injections were conducted prior to ethanol self-administration sessions to habituate subjects to the microinjection procedure. Next, ibogaine (0.1 μM/0.5 μl, 1 μM/0.5 μl and 10 μM/0.5 μl) or αCSF (0.5 μl) was injected into the VTA of gently restrained rats at a rate of 0.1 μl/minute via an internal injection cannula extending 0.5 mm beyond the guide cannula tip. Injection cannulae were left in place for an additional 2 minutes to allow for diffusion of the drug. Three, 24 and 48 hours post injection, rats were placed into the self-administration chambers for a 1-hour self-administration session.

Minipump Implantation for Delivery of Anti-GDNF Neutralizing Antibodies

After ethanol self-administration was established as described above, bilateral osmotic pump guide cannulae (Plastics One) were implanted into the VTA (5.6 mm posterior to bregma, 1.0 mm mediolateral, 8.5 mm ventral to the skull surface). After 7 days recovery, subjects were given 10-15 training sessions to allow recovery of baseline levels of ethanol self-administration (rats in the control group were consuming 0.65±0.11 g/kg, while rats in the experimental group were consuming 0.66±0.08 g/kg). An osmotic minipump (Alzet) was then connected to each of the two guide cannulae and was implanted subcutaneously for continuous infusion of either anti GDNF neutralizing antibodies (600 ng/12 N^{o}l/side/day) or mouse IgG (600 ng/12 μl/side/day). Two days later, 1-h daily ethanol self-administration sessions resumed for 10 days. On the ₈ₜₕday, rats received an i.p. injection of 40 mg/kg ibogaine.

Conditioned Place Preference Test (CPP)

The CPP apparatus (Med Associates) consisted of two visually distinctive conditioning compartments connected by a smaller center compartment. The CPP procedure consisted of three phases: preconditioning (Day 1), conditioning (Days 2-9), and post-conditioning (Day 10). On Day 1, mice were placed into the center compartment and allowed free access to both conditioning compartments for 30 minutes. To test whether ibogaine is aversive or rewarding, on Day 2, mice received i.p. injections of either saline or 40 mg/kg ibogaine. Mice were given access to one conditioning chamber for 5 minutes immediately after injection or 3 h after injection. The following day, subjects received ibogaine or saline (whichever they did not receive the day before), and were allowed access for 5 minutes to the chamber not paired with injection the day before. This daily alternation occurred until subjects received 4 pairings each of ibogaine and saline with their respective conditioning chambers. Control subjects received saline in both conditioning chambers. To test whether ibogaine alters ethanol-induced CPP, on Day 2, ibogaine (40 mg/kg, i.p.), or saline, was injected 3 hours prior to injection of ethanol (2 g/kg, 20% solution). Ethanol treatment was followed by placement within one conditioning chamber, alternated with saline treatment and placement into the other conditioning chamber, as described above. On Day 10, animals were tested for CPP by allowing free access to both conditioning compartments for 30 minutes. Photocell counts provided a measure of locomotor activity as well as time spent in each compartment

Histology

Locations of cannulae were verified in 50 N^{o}m sections stained with thionin. Only data from subjects with cannulae located in the VTA were included in the analyses. For the microinjection experiment, the tips of the injection cannulae were found to be 4.5 to 6 mm posterial bregma, 0.8 to 1.2 mm mediolateral and 8.2-8.8 mm ventral to the skull surface. For the minipump experiment, the tips of the infusion cannulae were found to be 4.8 to 6.3 mm posterial bregma, 0.8 to 1.2 mm mediolateral and 8-8.8 mm ventral to the skull surface.

Data Analysis

Student's t-test was used to evaluate differences in in vivo gene expression, and for the biochemical and CPP behavioral data. Two-bottle preference measures, locomotor activity measures, and ethanol self-administration data were analyzed by two-way ANOVA with repeated measures. F-values attaining significance were evaluated further by Student-NewmanKeel method. P<0.05 was regarded as statistically significant.

Results

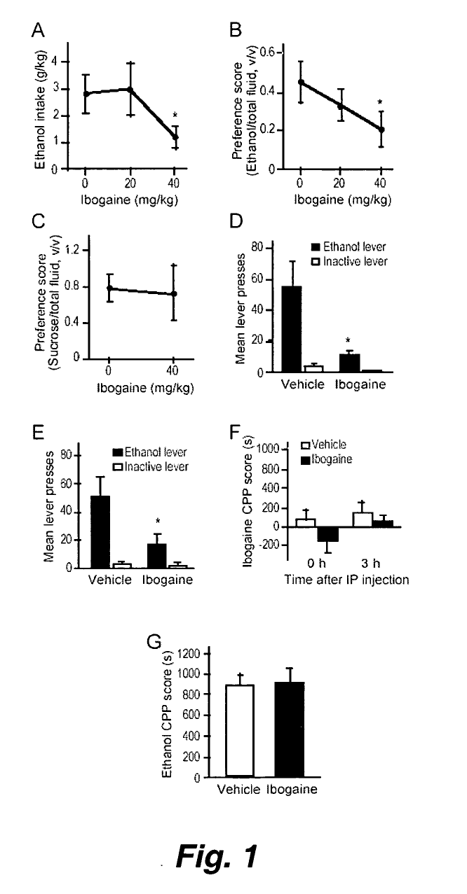

We first set to confirm that ibogaine administration attenuates ethanol self-administration. First, we tested the effects of systemic injection of 40 mg/kg ibogaine on voluntary ethanol consumption in rats allowed continuous access to ethanol in the home cage. This dose of ibogaine has been previously reported to reduce cocaine and heroin self-administration (Cappendijk and Dzoljic (1993) Eur. J. Pharmacol. 241: 261-265; Glick et al. (1992) Neurophannacology 31: 497-500; Dworkin et al. (1995) Psychopharmacology (Berl) 117: 257-261). This dose of ibogaine is not toxic to cells as it was previously shown not to produce Purkinje cell death (Molinari et al. (1996) Brain Res., 737: 255-262). In addition, we found no evidence of neuronal cell death upon systemic intra-peritoneal (i.p.) administration of ibogaine, as measured by staining 25 μm brain slices with Fluorolade, an anionic dye that causes dying and dead neurons to fluoresce bright green while living cells do not (Schmued et al. (1997) Brain Res., 751: 37-46). No differences in staining between control and ibogaine-treated sections were observed (data not shown). Under conditions of continuous access to ethanol in the home cage, ibogaine reduced both intake (FIG. 1, panel A) and preference (FIG. 1, panel B) for ethanol without an associated decrease in water intake (data not shown). No effect of ibogaine on preference for sucrose was observed (FIG. 1, panel C). Next, we tested the activities of i.p. administration of 40 mg/kg ibogaine in an ethanol operant self-administration paradigm. Ibogaine injection 3 hours prior to the behavioral session decreased responding at the ethanolpaired lever, and had no effect on inactive lever responding (FIG. 1, panel D). We also tested the effect of ibogaine in an ethanol reinstatement paradigm. As shown in FIG. 1, panel E, ibogaine reduced reinstatement of lever pressing for ethanol induced by response-contingent ethanol after a period of extinction.

The effects of ibogaine within the conditioned place preference (CPP) task in mice were determined to test whether ibogaine itself has aversive or rewarding properties. No significant ibogaine-conditioned preference or aversion was found, whether ibogaine was injected immediately or 3 hours before placement into the conditioning chambers (FIG. 1, panel F). Also, the rewarding properties of ethanol were not altered regardless of whether ibogaine was repeatedly injected during CPP acquisition (FIG. 1, panel G), injected 3 hours before CPP expression (data not shown), or both (data not shown). In addition, there was no change in the locomotor activity of mice after injection of ibogaine alone [F(7,21)=0.136, P=0.72] or when administered together with ethanol [F(7,21)=5.28, P=0.06] as measured within the place preference apparatus.

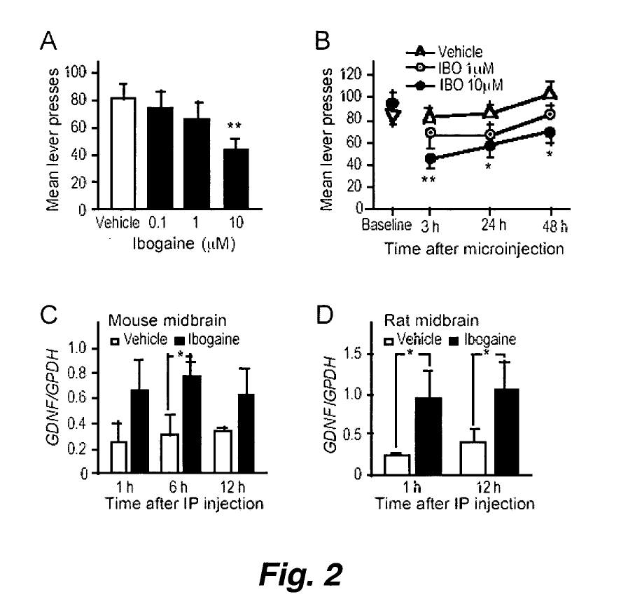

Next, we tested whether ibogaine directly acts within the VTA to inhibit ethanol selfadministration. Microinjection of ibogaine (0.1 μM/0.5 μl, 1.0 μM/0.5 μl and 10.0 μM/0.5 μl) into the VTA 3 hours before the lever pressing session, significantly decreased ethanol-paired lever pressing in a dose-dependent manner (FIG. 2, panel A), but did not affect the pressing of the inactive lever (data not shown). Interestingly, intra-VTA activities of ibogaine were found to be long-lasting since lever presses for ethanol did not return to baseline levels within 48 hours of injection of the 10 μM concentration of ibogaine (FIG. 2, panel B).

To identify the molecular mechanism that mediates the effects of intra-VTA ibogaine on ethanol intake, we used a small-scale expression array analysis (R&D) to study gene expression in the midbrain of mice that were injected with ibogaine. Interestingly, the same dose of ibogaine (40 mg/kg, i.p.) that caused a reduction in ethanol intake (FIG. 1, panels A-D), and ethanolinduced reinstatement (FIG. 1, panel E), also caused an increase in the mRNA expression of GDNF (data not shown). Next we confirmed these results by measuring the mRNA level of GDNF in mice and rats, at various time points after systemic administration of ibogaine. lbogaine administration caused an increase in the expression of GDNF in the midbrain in both mice (FIG. 2, panel C) and rats (FIG. 2, panel D).

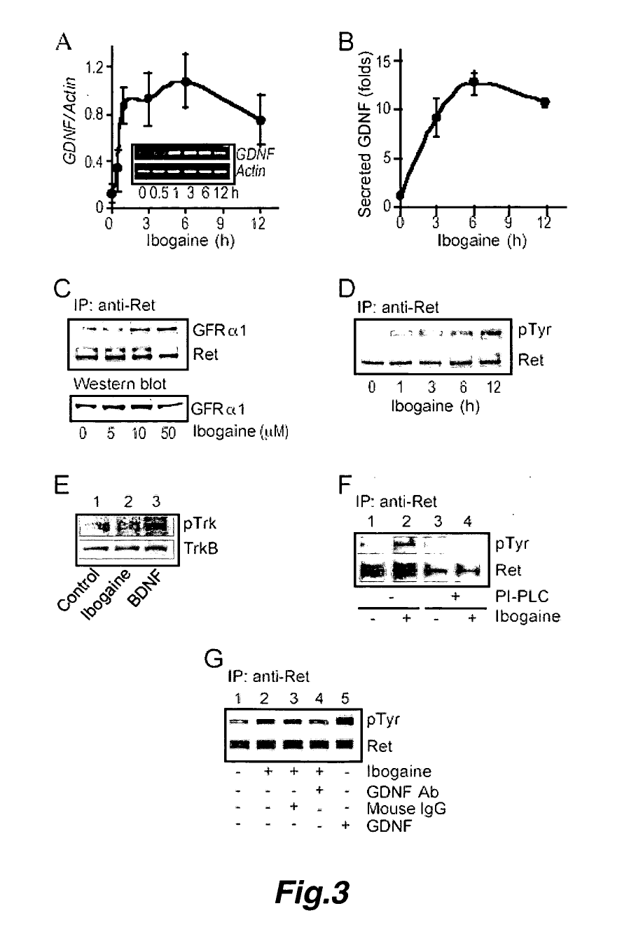

GDNF has been previously shown to be expressed in the midbrain region (Pochon et al. (1997) Eur. J. Neurosci., 3: 463-71, Semba et al. (2004) Brain Res. Mol. Brain Res., 124: 88-95). The GDNF receptors GFRα1 and Ret are mainly expressed in dopaminergic neurons, especially the VTA (Glazer et al. (1998) J. Comp. Neurol. 391: 42-49; Sarabi et al. (2001) J. Comp. Neurol., 441: 106-117). Previous studies by Messer et al. found that infusion of GDNF into the VTA reversed several biochemical and behavioral adaptations to repeated exposure to morphine and cocaine (Messer et al. (2000) Neuron 26: 247-257). Therefore, we hypothesized that ibogaine reduces ethanol consumption by activating the GDNF pathway. To test this hypothesis, we used the dopaminergic SHSY5Y human neuroblastoma cell line as a cell culture model and tested the activities of ibogaine on GDNF expression, secretion and signaling. Ibogaine induced a dose-dependent (data not shown) and time-dependent (FIG. 3, panel A) increase in GDNF expression that lasted up to 12 hours. Next, we examined whether the increase in GDNF mRNA levels leads to an increase in GDNF secretion. To do so, we measured GDNF levels in the media of cells treated with ibogaine, and found that GDNF accumulated in the media in a time-dependent manner consistent with the time course for mRNA increases (FIG. 3, panel B).

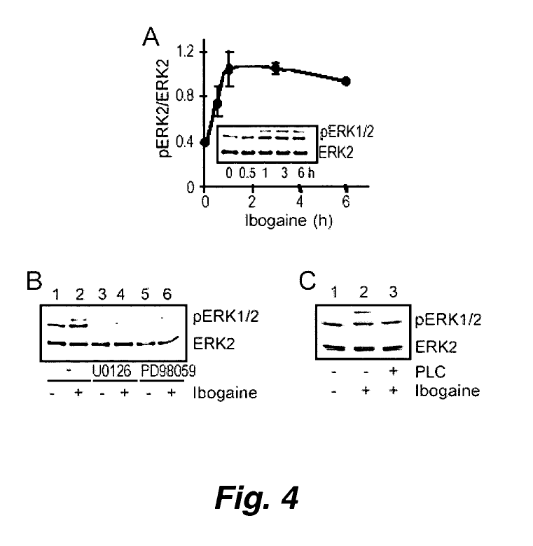

Activation of the GDNF pathway is initiated upon the ligation of GDNF with GFRα1 leading to the association of Ret with GFRα1, and the consequent auto-phosphorylation, and thus activation, of Ret (Jing et al. (1996) Cell 85: 1113-1124; Treanor et al. (1996) Nature 382: 80-83). Hence, we assessed whether ibogaine induces the association of Ret with GFRα1. Ibogaine treatment increased Ret association with GFRα1 (FIG. 3C, top panel), and this increased association was not due to increased GFRα1 protein levels (FIG. 3, panel C, bottom panel). Next, we determined whether the ibogaine-induced association of Ret with GFRα1 leads to the activation of the GDNF pathway by measuring the levels of phospho-Ret in the absence or presence of ibogaine. Tbogaine induced Ret phosphorylation in a dose- (data not shown) and time-dependent manner (FIG. 3, panel D). We found no change in the phosphorylation state of another growth factor receptor tyrosine kinase, Trk in the presence of ibogaine (FIG. 3, panel E). GFRα1 is a glycosyl-phosphatidylinositol (GPI)anchored protein that can be hydrolysed by PI-PLC (Jing et al. (1996) Cell 85: 1113-1124; Treanor et al. (1996) Nature 382: 80-83). If ibogaine activates Ret by inducing the association of Ret with GFRα1, then ibogaine-induced Ret phosphorylation should be prevented by PI-PLC. Pre-incubation of the cells with PI-PLC abolished ibogaine-induced Ret phosphorylation (FIG. 3, panel F). Ret phosphorylation induced by ibogaine was also inhibited in the presence of the anti-GDNF neutralizing antibodies (FIG. 3, panel G, lanes 2 vs. 4). Finally, we determined whether down-stream signaling cascades known to be activated by GDNF (Trupp et al. (1999) J. Biol. Chem. 274: 20885-20894; Hayashi et al. (2000) Oncogene 19: 4469-4475) were also activated in the presence of ibogaine. We measured the phosphorylation, and thus activation state, of ERK1/2, also named p42/44 mitogen-activated protein kinases (p42/44 MAP kinases). We found that ibogaine induced the phosphorylation of ERK1/2, and the activation lasted up to 6 hours (FIG. 4, panel A). Furthermore, two inhibitors, U0126 and PD98059, that are specific for MAPK/ERK kinase 1 (MEK1), a kinase upstream of ERK1/2, blocked ERK1/2 phosphorylation induced by ibogaine (FIG. 4, panel B). Finally, pre-incubation with PI-PLC abolished ibogaine-induced ERK1/2 phosphorylation (FIG. 4, panel C), suggesting that ibogaine-induced ERK1/2 phosphorylation is mediated by the association of the GDNF receptors GFRα1 and Ret.

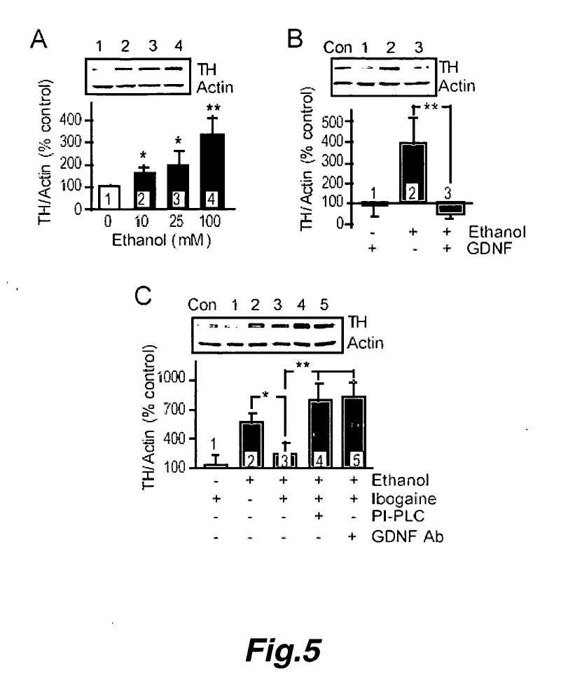

If the activation of the GDNF pathway mediates some of the “desirable” properties of ibogaine, then the drug should reverse biochemical adaptations induced by prolonged exposure to ethanol. We tested this hypothesis by determining the effects of ibogaine on the protein levels of tyrosine hydroxylase (TH), the rate-limiting enzyme for dopamine synthesis. Altered levels of TH in the brain is a hallmark of the biochemical adaptation to drugs of abuse including ethanol (Beitner and Nestler (1991) J. Neurochem., 57: 344-347.; Vrana et al. (1993) J. Neurochem. 61: 2262-2268; Ortiz et al. (1995) Synapse 21: 289-298). We found that exposure of SHSY5Y cells to ethanol for 24 hours produced a significant increase in the protein level of TH in a dose-dependent manner (FIG. 5, panel A). Treatment of SHSY5Y cells with ethanol concentrations as low as 10 mM resulted in an increase in TH immunoreactivity, and this increase was inhibited by the addition of GDNF (FIG. 5, panel A, lanes 2 vs. 3) or ibogaine (FIG. 5, panel B, lanes 2 vs. 3). Ibogaine's activities were inhibited when cells were incubated with either PI-PLC or anti-GDNF neutralizing antibodies (FIG. 5, panel B, lane 3 vs. lanes 4 and 5), suggesting that ibogaine reverses the increased levels of TH induced by ethanol via GDNF.

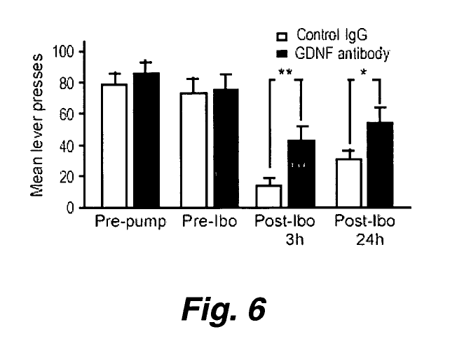

Because intra-VTA ibogaine decreased ethanol self-administration, and because ibogaine administration increased GDNF expression in the midbrain, which includes the VTA, we tested whether anti-GDNF neutralizing antibodies (Messer et al. (2000) Neuron 26: 247-257) infused into the VTA would attenuate the ibogaine-induced decrease in ethanol self-administration. Two subcutaneous osmotic mini-pumps were attached to bilateral cannulae aimed at the VTA to continuously infuse either anti-GDNF neutralizing antibodies or control mouse IgG for 2 weeks. During this time, ethanol intake was measured in daily operant-self administration sessions. Neither the implantation itself, the infusion of vehicle (data not shown), nor the infusion of control mouse IgG altered ethanol self-administration (FIG. 6). However, infusion of anti-GDNF neutralizing antibodies significantly attenuated the ability of ibogaine to decrease ethanol self-administration both 3 h and 24 h after ibogaine administration (FIG. 6). Specifically, 3 hours after ibogaine injection, control subjects decreased responding for ethanol by 79.1% (±0.05), while only a 39.1% (±0.13) decrease was observed in animals receiving the anti-GDNF neutralizing antibodies (FIG. 6). However, the inactive lever pressing was unchanged (data not shown).

Discussion

We found that ibogaine reduces ethanol consumption and ethanol-induced reinstatement in rats, and that this effect is likely due to ibogaine actions within the VTA, as microinjection of ibogaine into the VTA also decreased ethanol self-administration. In addition, systemic administration of ibogaine specifically increased the expression levels of GDNF in the VTA. In a cell culture model, we further found that the ibogaine increase in expression of GDNF led to increased secretion of GDNF and to the subsequent activation of the GDNF pathway. Ibogaine's activation of the GDNF pathway blocked ethanol-induced increases in TH protein levels. Finally, we found that ibogaine's activities were diminished when it was given systemically in the presence of anti-GDNF neutralizing antibodies infused into the VTA. These findings collectively suggest that ibogaine via increasing the level of GDNF in the VTA blocks the biochemical and behavioral adaptations to long-term exposure to ethanol.

A previous study found that ibogaine decreased ethanol consumption in ethanolpreferring rats in the two-bottle preference paradigm (Rezvani et al. (1995) Pharmacol. Biochem. Behav., 52: 615-620). Our initial studies were designed to replicate these findings in an outbred line of rats, and to extend these findings to the operant self-administration procedure. In agreement with Rezvani et al. (1995) we found that ibogaine in a similar dose range decreases ethanol intake whether the ethanol is available continuously on the home cage, or whether it is available upon lever press responding for a limited time each day. Ibogaine was also effective in reducing ethanol intake the first day access was again granted after a period of extinction; this reinstatement of previously extinguished responding may be analgous to the avid ethanol consumption seen among human alcoholics following relapse. Interestingly, human anecdotal reports also suggest a decrease in craving and relapse to drugs of abuse after intake of ibogaine (Mash et al. (1998) Ann. N. Y. Acad. Sci., 844: 274-292). Taken together, these findings indicate that ibogaine reduces ethanol intake suggesting that the identification of its molecular mechanism of action for this effect may be beneficial.

It is unlikely that at the dose of ibogaine used for the experiments (40 mg/kg) the reduction of ethanol intake is due to non-specific activities of the drug for several reasons. First, we found that the administration of ibogaine does not cause conditioned place aversion in mice. Second, we found that ibogaine did not reduce preference for a sucrose solution. This finding is in agreement with a previous study that found no effect of 40 mg/kg ibogaine on intake of a sucrose/saccharin solution in rats (Blackburn and Szumlinski (1997) Behav. Br. Res. 89:99-106). Second, it is unlikely that changes in locomotor activity can explain the decrease in ethanol consumption. Although it has been reported that ibogaine alters locomotor activity in rats within one hour of injection, both decreases (Kesner et al. (1995) Phannacol. Biochem. Behav., 51: 103-109) and increases (Baumann et al. (2001) J. Pharmacol. Exp. Ther. 297: 531-539) were observed. In our experiments, we investigated the effects of ibogaine on ethanol intake 3 hours after ibogaine injection. We found that ibogaine does not alter locomotor activity in mice at this time point. Our result in mice are in agreement with a previous report in rats showing that ibogaine itself does not affect locomotor activity between 3 to 4 hours post-injection (Maisonneuve et al. (1997) J. Pharmacol. 336: 123-126). In addition, we found no effect of ibogaine on inactive lever responding in the operant self-administration procedure. While low responding on the inactive lever precludes examination of non-specific decreases in activity, these findings suggest that ibogaine did not produce nonspecific increases in activity. Finally, we found no evidence for ibogaine-induced neurotoxcitiy in the brain. This result is in concordance with a study by Molinari at el. (1996) that found noe vidence for neuronal cell death after 40 mg/kg injection (Molinari et al. (1996) Brain Res., 737: 255-262). Hence, it is unlikely that ibogaine's effects on ethanol intake in these studies resulted from a neurotoxic effect.

One possible explanation for the reduction of ethanol intake by ibogaine is that ibogaine might substitute for ethanol. However, ibogaine administration itself did not produce a place preference, suggesting that it is not rewarding. We also found that ibogaine did not alter the rewarding properties of ethanol in the CPP procedure. It remains to be determined if higher doses of ibogaine would be effective in the CPP procedure. Alternatively, it may be that ibogaine affects ethanol consumption, but not ethanol conditioned reward.

In the first part of the study, we confirmed that ibogaine reduces ethanol intake. However, because ibogaine is a hallucinogen, it is not useful for treatment. Therefore, in the second part of our study we set out to determine a molecular mechanism of action of ibogaine that mediates its reduction of ethanol self-administration. We reasoned that ibogaine might alter signaling pathways within the “addiction” neurocircuitry. We found that microinjection of ibogaine into the VTA reduced ethanol self-administration intake in an operant selfadministration paradigm. Interestingly, we also found that systemic administration of ibogaine to both mice and rats increased the expression of GDNF in the midbrain, a region that includes the VTA. Induction of GDNF expression in the midbrain dopaminergic neurons has been previously reported after treatment with phencyclidine (Semba et al. (2004) Brain Res. Mol. Brain Res., 124: 88-95).

We further found that, in SHSY5Y cells, the ibogaine-induced increase in expression of GDNF led to increased secretion of GDNF and to the subsequent activation of the GDNF pathway. Because SHSY5Y cells are dopaminergic neuronal-like cells (Kaplan et al. (1993) Neuron 11: 321-331; Encinas et al. (2000) J. Neurochem. 75: 991-1003), it is possible that the source of GDNF is neuronal. However, since GDNF is expressed in both glia and neurons (Airaksinen and Saarma (2002) Nat. Rev. Neurosci., 3: 383-394), at this point, we cannot exclude the possibility that ibogaine increases the expression of GDNF in astrocytes.

ibogaine induction of GDNF expression lasted for at least 12 hours both in cultured cells and in vivo. In addition, ibogaine's effects on ethanol self-administration were long lasting as well. The half-life of ibogaine is reported to be about 1-2 hours, while its metabolite, Noribogaine, is detected at high concentrations within 24 hours after administration (Mash et al. (2000) Ann. N.Y. Acad. Sci. 914: 394-401; Glick et al. (1992) Neurophantnacology 31: 497-500). It is therefore possible that Noribogaine maintains the long-lasting expression of GDNF in the brain. Another possibility is that the initial increase in GDNF expression induced by ibogaine is followed by autocrine regulation of the growth factor's expression.

Importantly, we found that ibogaine's activities were diminished when it was given systemically in the presence of anti-GDNF neutralizing antibodies infused into the VTA. Hence, decreasing levels of GDNF in the VTA decreases ibogaine's effect on ethanol consumption. This finding supports the hypothesis that ibogaine reduces ethanol intake by increasing the expression and subsequent activity of GDNF. Previous studies documented GDNF as a molecule that counteracts the effects of prolonged exposure to drugs of abuse including ethanol. For example, infusion of GDNF into the VTA reversed morphine-induced increases in protein levels of TH (Messer et al. (2000) Neuron 26: 247-257). Messer et al. (2000) also reported that intra-VTA GDNF treatment blocked and reversed the biochemical effects of cocaine and blocked the behavioral effects of repeated exposure to cocaine measured by the conditioned place preference test. GDNF was also found to prevent ethanol-induced apoptosis in a neuroblastoma cell line (McAlhanyet al. (2000) Brain Res. Dev. Brain Res., 119: 209-216). Conversely, chronic exposure to drugs of abuse and ethanol decrease GDNF signaling. Chronic cocaine and chronic morphine exposure decreased the levels of Ret phosphorylation in the VTA (Messer et al., 2000, supra.). Prolonged exposure to ethanol decreased GDNF secretion in developing cerebellum (McAlhany et al. (1999) Alcohol Clin. Exp. Res. 23: 1691-1697), and prenatal exposure to cocaine resulted in a decrease in GDNF levels (Lipton et al. (1999) Brain Res. Dev. Brain Res., 118: 231-235). Because exogenous administration of GDNF blocks the effects of repeated administration of drugs of abuse, it is plausible that endogenous GDNF systems may serve to counter the effects of these drugs, and that ibogaine acts to inhibit intake of stimulants, opiates, and ethanol by enhancing this protective homeostatic pathway.

ibogaine increased the activity of the MAP kinase pathway, which has been reported to be activated via GDNF (Hayashi et al. (2000) Oncogene 19: 4469-4475; Besset et al. (2000) J. Biol. Chem., 275: 39159-39166). Interestingly, activation of the MAP kinase pathway was previously shown to result in the inhibition of A-type potassium channels (Yuan et al. (2002) J. Neurosci. 22: 4860-4868), and GDNF has been recently reported to enhance the excitability of midbrain dopaminergic neurons by inhibiting the A-type potassium channels (Yang et al. (2001) Nat. Neurosci., 4: 1071-1078). Chronic ethanol inhibits the excitability of VTA neurons (Bailey et al. (1998) Brain Res. 803: 144-152), and the fling rates of dopaminergic cells are markedly reduced in the early abstinence period after chronic ethanol consumption (Bailey et al. (2001) Neuropharmacology 41: 989-999). These findings suggest that ibogaine, via GDNF, may reduce the actions of ethanol in the VTA by inhibiting the activity of A-type potassium channels, thus enhancing VTA firing.

Prolonged exposure of SHSY5Y cells to ethanol resulted in a marked increase in the protein levels of TH, which was blocked by ibogaine via GDNF. Interestingly, Schmidt et al. reported that the level of TH was significantly increased in the VTA following extinction of operant responding for cocaine (Schmidt et al. (2001) J. Neurosci., 21: RC137), and we found that ibogaine decreased ethanol-induced reinstatement after a period of extinction, possibly by reducing the levels of TH.

Therefore, it is possible that ibogaine reduces ethanol consumption by increasing the levels of GDNF in the VTA and by activating the GDNF pathway to reduce the action of ethanol on proteins such as TH.

In conclusion, we have identified GDNF as a candidate molecule that mediates, at least in part, the activities of ibogaine on ethanol consumption. Therefore, this study may lead to a promising new approach for the treatment of drug and alcohol abuse by the development of agents that up-regulate the GDNF pathway.

### Example 2

### RACK1 and BDNF: A Homeostatic Pathway that Regulates Alcohol Addiction

Alcoholism is a devastating disease that manifests as uncontrolled drinking. Consumption of alcohol is regulated by neurochemical systems within specific neural circuits, but endogenous systems that may counteract and thus suppress the behavioral effects of ethanol including intake are unknown.

Here we tested the possibility that BDNF is part of a homeostatic pathway that regulates ethanol intake. We also assessed the interactions between RACK1, BDNF, and ethanol, and determined whether the RACK1/BDNF pathway is involved in the regulation of the behavioral actions of ethanol.

In this example, we demonstrate that BDNF reduces the behavioral effects of ethanol, including consumption, in rodents. We found that decreases in BDNF levels or signaling results in increased behavioral responses to ethanol whereas increases in the levels of BDNF, mediated by the scaffolding protein RACK1, attenuates these behaviors. Interestingly, we found that acute exposure of cultured neurons to ethanol leads to increased levels of BDNF mRNA via RACK1. Importantly, voluntary ethanol consumption also leads to increased levels of BDNF expression. Taken together, these findings suggest that RACK1 and BDNF are part of a homeostatic pathway that opposes adaptations that maintain addiction.

Results

Ethanol Increases BDNF Expression

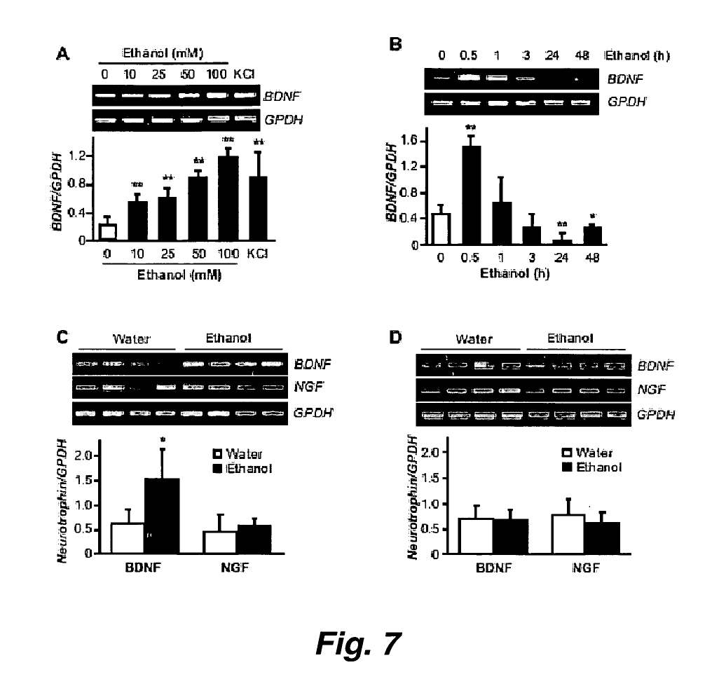

If BDNF is part of a homeostatic pathway that counters ethanol effects, then it should be upregulated upon exposure to ethanol. Therefore, we examined whether acute exposure to ethanol alters the expression of BDNF by reverse transcriptase-polymerase chain reaction (RT-PCR). We found that ethanol treatment increased the expression levels of BDNF in primary hippocampal neurons in a dose- and time-dependent manner (FIG. 7, panel A and panel B). Concentrations as low as 10-25 mM, that correspond to blood alcohol concentrations following moderate consumption by humans (Eckardt et al. (1998) Alcohol: Clin. Exp. Res., 22: 998-1040), increased BDNF expression (FIG. 7, panel A). Interestingly, the increase in BDNF expression was biphasic, since acute exposure to ethanol resulted in an increase in BDNF mRNA levels in neurons, whereas continuous exposure to ethanol for 24 or 48 hours, reduced BDNF expression to below basal levels of untreated cells (FIG. 7, panel B).

Next we determined whether the increase in BDNF expression by ethanol is observed in vivo following self-administration of ethanol. C57 mice were allowed continuous, unlimited access to both water and 10% ethanol in their home cage for 2 weeks. Mouse brain regions were taken 3 hours after the start of the dark cycle—a time period of high ethanol consumption (Freund (1970) J. Nutr., 100: 3-36)—and analyzed for BDNF and NGF mRNA. Consumption of ethanol significantly increased the expression levels of BDNF in the striatum (FIG. 7, panel C), but not the prefrontal cortex (PFC) (FIG. 7, panel D). The increase in consumption was specific for BDNF since no change was observed in expression of the nerve-derived growth factor (NGF), a closely related neurotrophic factor (FIG. 7, panels C and D).

BDNF Regulates the Behavioral Effects of Ethanol

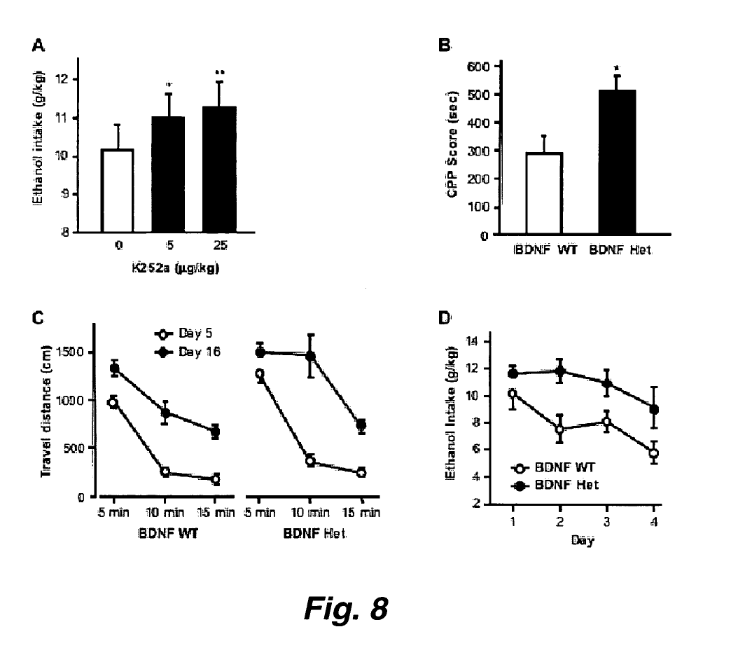

We hypothesized that the ethanol-mediated increase in BDNF contributes to a homeostatic pathway that counteracts the neurochemical systems responsible for the escalation and maintenance of ethanol consumption. If this is so, then inhibition of the BDNF signaling pathway also should result in increased ethanol intake. We therefore determined the effects of the Trk kinase inhibitor K252a (Tapley et al. (1992) Oncogene 7: 371-381) on voluntary ethanol intake in wildtype mice. As shown in FIG. 8, panel A, K252a increased ethanol consumption [main effect of treatment F(2,40)=6.18, P<0.006] at both 5 mg/kg (P<0.02) and 25 mg/kg (P<0.005), but water consumption was not affected (mis consumed, mean±SEM: vehicle, 1.72±0.29; 5 mg/kg, 1.69±0.28; 25 mg/kg, 1.95±0.27). K252a is not a specific inhibitor of the BDNF TrkB receptor; it also can affect other Trk receptors. However, since the levels of NGF in the presence of ethanol were not altered, it is likely that the increase in voluntary ethanol intake upon K252a administration is mediated via the inhibition of BDNF signaling cascade.

To test whether the effects of BDNF on the behavioral response to ethanol are limited to oral self-administration, we examined the ability of BDNF heterozygote (BDNF±) mice and their litter-mate wild-type controls to form an association between a specific environment and the rewarding effects of repeated intraperitoneal (i.p.) ethanol injection. We found that decreasing the levels of BDNF by 50% results in significantly greater preference for the environment paired with ethanol injection than that observed in wildtype control mice (FIG. 8, panel B) [F(1,36)=5.81, P<0.03]. We also tested BDNF±mice for behavioral sensitization of the locomotor stimulatory effects of ethanol, a model for progressive changes in drug responsiveness that may underlie enhanced motivational effects of the drug (Robinson and Berridge (1993) Brain Res. Rev. 18: 247-291). BDNF±mice and wildtype mice did not differ in their baseline levels of locomotor activity after a saline injection (BDNF+/+mice, 1872.04 cm±126.90; BDNF±mice, 1982.07 cm±133.37), but mice lacking one copy of the BDNF gene demonstrated a greater initial locomotor effect of ethanol injection during the 15 min test session on Day 5 [Main effect of genotype: F(1,22)=9.01, P<0.01]. Both BDNF+/+ and BDNF±mice developed ethanol sensitization as indicated by an increase in distance traveled following the ethanol injection on Day 16 as compared to Day 5 (FIG. 8, panel C) [Main effect of day, BDNF+/+: F(1,13)=47.77, P<0.001; BDNF±: F(1,9)=40.49, P<0.001]. However, the increase in locomotor activity on Day 16 was enhanced in BDNF±mice as indicated by a significant day x time interaction [F(2,18)=10.71, P<0.001] for the BDNF±mice when comparing activity on Day 5 and Day 16. In addition, a comparison of the distance traveled by the BDNF+/+ and BDNF±mice on Day 16 revealed a significant genotype×time interaction [F(2,44)=4.71, P<0.02] that is accounted for by an enhanced locomotor response to the ethanol challenge at the 10 min time point in the BDNF±mice as compared to wildtype control mice (P<0.002; FIG. 8, panel C). Therefore, reduced BDNF is associated with enhanced locomotor stimulant effects of acute and repeated ethanol, indicating that BDNF influences neural systems, likely the VTA-NAC circuitry (Phillips and Shen, 1996), that mediate the initial locomotor effects of ethanol as well as sensitized locomotor effects. Importantly, both the place conditioning and the locomotion studies indicate that BDNF may directly alter systems that mediate the addictive properties of ethanol, regardless of the route of administration of ethanol. These results also suggest that BDNF does not modulate ethanol intake by affecting food regulation (Kernie et al. (2000) EMBO J., 19: 1290-1300; Rios et al. (2001) Mol. Endocrinol. 15: 1748-1757).

We also determined whether BDNF±mice and their litter-mate wild-type controls differ in their voluntary ethanol consumption after a deprivation period of 2 weeks. Decreasing the levels of BDNF by 50% results in a significant increase in ethanol intake as indicated by a main effect of genotype [F(1,16)=6.50, P<0.03] (FIG. 8, panel D), suggesting that BDNF also controls consumption after a period of abstinence.

Together, these results suggest that ethanol-induced increases in BDNF regulate the behavioral response to ethanol, and that the BDNF pathway may therefore protect against deleterious behavioral effects of ethanol, including intake.

BDNF Expression is Mediated by RACK1

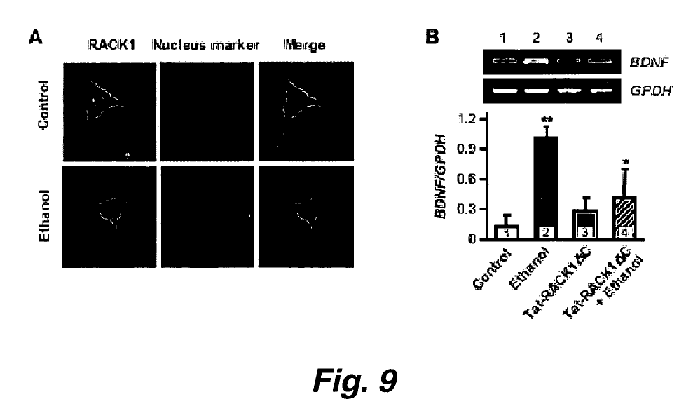

We previously found that acute ethanol treatment causes the translocation of the scaffolding protein RACK1 to the nucleus via the activation of the cAMP dependent protein kinase A (PKA) (Ron et al. (2000) FASEB J. 14: 2303-2314). In the nucleus, RACK1 is involved in the regulation of expression of several genes (He et al. (2002) Mol. Pharm. 62: 272-280) including BDNF (Yaka et al. (2003) J. Biol. Chem. 278: 9630-9638). We therefore hypothesized that acute ethanol treatment increases the expression of BDNF by stimulating RACK1 translocation to the nucleus. To test this hypothesis, we first verified that ethanol treatment results in the translocation of RACK1 into the nucleus of primary hippocampal neurons (FIG. 9, panel A). Next, we measured BDNF expression in the presence of ethanol and a dominant negative fragment of RACK1, RACK1ΔC, expressed as a Tat-fusion protein (Tat-RACK1ΔAC). Transduction of Tat-RACK1ΔC pevents RACK1 nuclear translocation and gene expression (He et al. (2002) Mol. Pharm. 62: 272-280; Yaka et al. (2003) J. Biol. Chem. 278: 9630-9638). We found that RACK1ΔC significantly inhibited ethanol induction of BDNF expression in hippocampal neurons (FIG. 9, panel B, lanes 2 vs. 4), indicating that nuclear translocation of RACK1 in response to acute ethanol mediates the increase in BDNF expression.

Tat-RACK1 Increases BDNF Expression

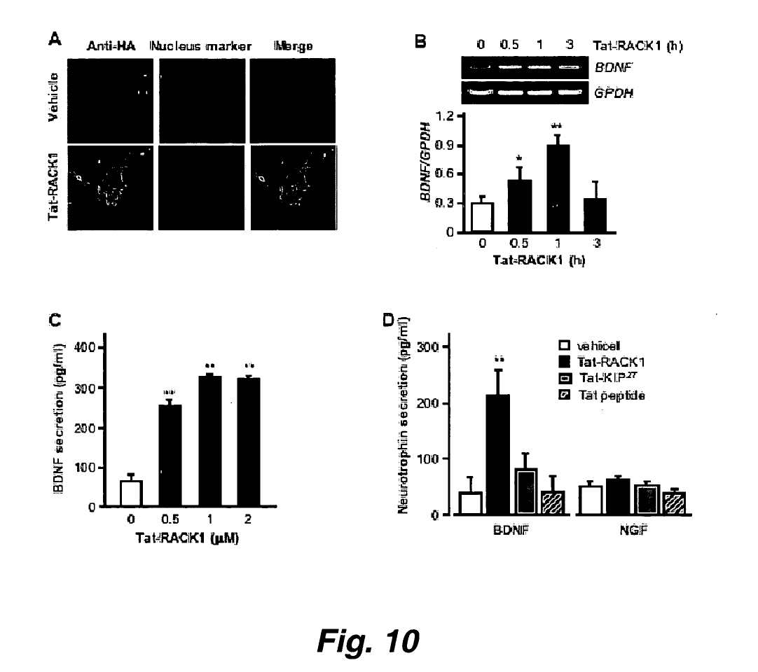

Thus far, we have shown that ethanol exposure results in an increase in the mRNA levels of BDNF via nuclear RACK1, and that BDNF is a negative regulator of the behavioral effects of ethanol. We reasoned that if the RACK1/BDNF pathway is important for controlling the behavioral response to ethanol, then elevation of expression levels and activity of this pathway in vivo should attenuate the response to ethanol. To test this hypothesis, we first tested whether increasing the protein levels of RACK1 results in elevation of BDNF message, protein, and signaling in reduced preparations. We incubated dissociated hippocampal neurons with recombinant RACK1 expressed as a Tat-HA-tagged fusion protein (Tat-RACK1) and measured BDNF mRNA levels and protein secretion. As shown in FIG. 10, panel A, 2 hours of incubation with Tat-RACK1 resulted in efficient transduction of the protein throughout the cell including the nucleus. These results are consistent with our previous studies in which we found that Tat-RACK1 is efficiently transduced in cultured cells and brain slices (He et al. (2002) Mol. Pharm. 62: 272-280; Yaka et al. (2003) J. Biol. Chem. 278: 9630-9638). Next, we found that incubation of neurons with Tat-RACK1 increased the mRNA levels of BDNF in a time- (FIG. 10, panel B) and dose- (data not shown) dependent manner. Incubation with Tat-RACK1 also resulted in a significant increase in the secretion of BDNF in a dose- (FIG. 10, panel C) and time- (data not shown) dependent manner. Tat-RACK1 induction of BDNF secretion was specific since no change in BDNF secretion was observed when hippocampal neurons were treated with Tat-KIP27, a Tat-fusion protein of a similar molecular weight and charge to Tat-RACK1, or the Tat-peptide itself, and Tat-RACK1 did not alter the secretion of NGF (FIG. 10, panel D).

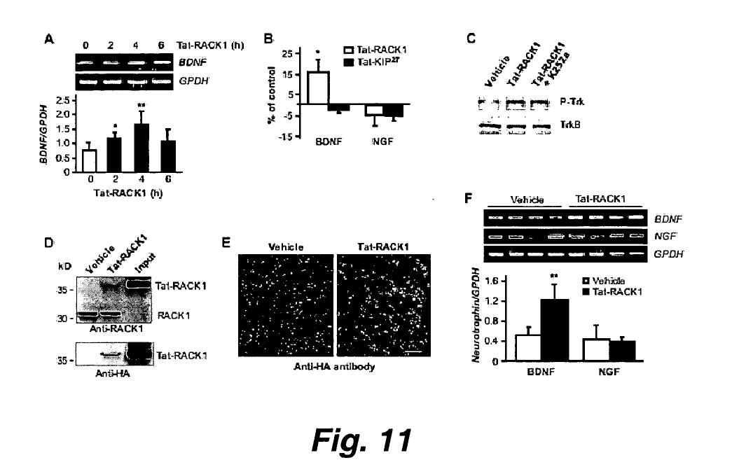

Since we found the expression level of BDNF to be elevated in the striatum (FIG. 7, panel C), and because BDNF alters gene expression in the striatum (Crol1 et al. (1994) Eur. J. Neurosci. 6: 1343-1353; Guillin et al. (2001) Nature 411: 8689), we determined whether Tat-RACK1 increases the levels of BDNF in rat striatal slices comprised of the dorsal striatum and the NAc, a brain region implicated in the reinforcing and addictive properties of ethanol (Janak et al. (1999) Brain Res., 817: 172-184; Koob et al. (1998) Neuron 21: 467476; Weiss and Porrino (2002) J. Neurosci. 22, 3332-3337). We found a time-dependent increase in mRNA of BDNF (FIG. 11, panel A) that corresponded with a specific increase in total BDNF but not NGF protein (FIG. 11, panel B) in striatal slices treated with Tat-RACK1. Next, we determined whether the increase in striatal BDNF protein results in the activation of the BDNF receptor, TrkB. BDNF binds to TrkB receptor kinase, causing receptor dimerization and autophosphorylation (Patapoutian and Reichardt (2001) Curr. Opin. Neurobiol., 11: 272-280). We found that incubation of striatal slices with Tat-RACK1 increased Trk phosphorylation (FIG. 11, panel C, lanes 1 vs. 2), however, when Tat-RACK1 was incubated together with the Trk kinase inhibitor K252a, the phosphorylation was reduced (FIG. 11, panel C, lanes 2 vs. 3). These results suggest that Tat-RACK1 treatment results in the activation of the BDNF pathway by an increase in the secretion of BDNF and the consequent activation of the TrkB receptor.

In vivo Injection of Tat-RACK1 Increases Striatal BDNF Expression

To determine whether Tat-RACK1 increases BDNF levels in vivo, the level of RACK1 in the brain was increased by i.p. injection of the fusion protein. Tat-RACK1 was successfully transduced into the brain and was detected 4 hours after injection (FIG. 11, panel D and 11, panel E), in agreement with Schwarze et al. (1999) Science, 285: 1569-1572, who reported the functional transduction of a Tat-b-gal protein as indicated by enzymatic activity of the protein in brain 4 hours after systemic injection. Importantly we found that i.p. injection of Tat-RACK1 to mice increased the mRNA levels of BDNF in the striatum (FIG. 11, panel F). Hence, Tat-RACK1 induces the expression BDNF in vivo.

Tat-RACK1 Reduces Ethanol Consumption and Sensitization

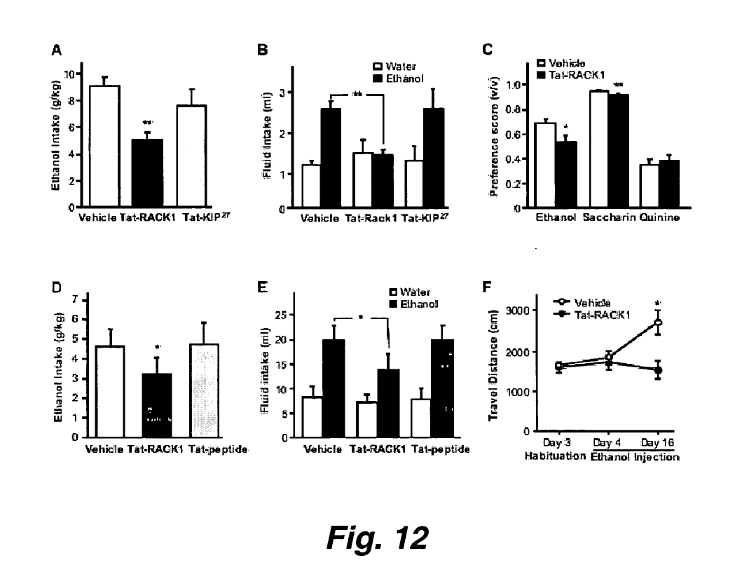

Noting both that ethanol induced an increase in BDNF expression via nuclear RACK1, and inhibition of the BDNF pathway increased ethanol intake and enhanced ethanol place preference and sensitization, we hypothesized that transient increases in brain levels of RACK1 that act to increase BDNF should reduce ethanol consumption. Therefore, we first tested the effects of Tat-RACK1 on voluntary ethanol intake by mice using a standard 2-bottle choice self-administration procedure. As predicted, Tat-RACK1 reduced ethanol consumption (FIG. 12, panels A-C). There was a significant decrease in ethanol intake relative to body weight [F(1,10)=48.40, P<0.001] (FIG. 12, panel A). In addition, analysis of the volume of ethanol and water consumed revealed a significant treatment x solution interaction [F(1,10)=9.54, P<0.02]. Post-hoc tests found that intake of ethanol was decreased after Tat-RACK1 injection (P<0.003) but water intake was not affected (FIG. 12, panel B). Preference for ethanol, measured as the proportion of volume of ethanol consumed relative to total fluid volume, was also reduced [F(1,10)=6.91, P<0.03] (FIG. 12, panel C). Injection of Tat-KIP27 did not alter ethanol intake (all P's>0.05), demonstrating specificity in the activity of Tat-RACK1 (FIG. 12, panels A and B). Control studies showed that Tat-RACK1 did not alter locomotor activity in mice, that body weight remained unchanged (data not shown), and that there was no significant change in quinine preference (FIG. 12, panel C), suggesting that Tat-RACK1 effects on ethanol intake are not mediated by general effects on locomotion, eating or taste reactivity. There was however a small (0.94 to 0.91, a 3 percentage point change), but statistically significant, reduction in saccharin preference after Tat-RACK1 injection [F(1,10)=15.13, P<0.005] (FIG. 12, panel C), suggesting that increases in Tat-RACK1 levels may reduce rewarding processes in general. To confirm that the behavioral actions of Tat-RACK1 are mediated via entry into the CNS, we microinjected the fusion protein directly into the CNS of rats. The intracerebroventricular (i.c.v.) administration of Tat-RACK1 reduced ethanol intake [F(1,7)=34.51, P<0.001] (FIG. 12, panel D). An examination of the volume of ethanol and water consumed revealed a main effect of treatment [F(1,7)=22.10, P<0.003] that is accounted for by a reduction in the volume of ethanol consumed (P<0.002), but no effect on the volume of water consumed (FIG. 12, panel E). There was no effect of i.c.v. injection of the Tat-peptide alone (all P's>0.05; FIG. 12, panels D and E). Taken together, these findings strongly suggest that Tat-RACK1 decreases ethanol intake, and that this effect is mediated by RACK1 actions within the CNS.

We used ethanol-induced behavioral sensitization to test the hypothesis that Tat-RACK1 reduces other behavioral effects of ethanol. We found that i.p. injection of Tat-RACK1 abolished the expression of ethanol-induced sensitization, as measured by decreased locomotor response to the challenge ethanol injection administered after 12 days of repeated ethanol treatment (FIG. 12, panel F) [main effect of treatment F(1,20)=4.82, P<0.05; main effect of day, F(1,20)=5.22, P<0.04; treatment×day interaction, F(1,20)=12.05, P<0.003; Day 16 vehicle vs. Tat-RACK1 treatment, P<0.002]. Control subjects that received saline instead of ethanol during both treatment and test reveal that Tat-RACK1 alone did not affect baseline locomotion (vehicle: 1546.76 cm±231.17, Tat-RACK1: 1323.86 cm±153.86). These results suggest that Tat-RACK1 prevents the expression of the increased responsiveness to ethanol that may underlie compulsive ethanol-seeking (Robinson and Berridge (1993) Brain Res. Rev. 18: 247-291). Taken together, these findings indicate that Tat-RACK1 decreases the effects of ethanol in multiple models of addiction, further strengthening the hypothesis that Tat-RACK1 is part of a mechanism that inhibits the behavioral effects of ethanol.

Tat-RACK1 Reduces Ethanol Intake via BDNF

If Tat-RACK1 decreases ethanol consumption by increasing the levels of BDNF, then changes in BDNF expression in vivo should alter Tat-RACK1's actions on ethanol intake. To test this hypothesis we determined the effects of Tat-RACK1 on ethanol consumption in BDNF±mice and their litter-mate wild-type controls. We hypothesized that these mice would have an attenuated response to Tat-RACK1 injection. As shown in FIG. 13, panel A, the effect of Tat-RACK1 on ethanol intake was decreased in BDNF±mice as compared to wild-type litter-mate control subjects [F(1,26)=4.74, P<0.04]. We propose that the reduction in Tat-RACK1 potency for decreasing ethanol consumption in the BDNF±mice reflects the decreased ability of Tat-RACK1 to induce BDNF expression, as there is only one copy of the BDNF gene. We also hypothesized that if Tat-RACK1 actions on ethanol self-administration are mediated via the BDNF signaling cascade, then the Trk receptor kinase inhibitor K252a should block Tat-RACK1 effects on ethanol intake. As predicted, co-injection of Tat-RACK1 with K252a prevented the reduction in ethanol intake seen after Tat-RACK1 alone (FIG. 13, panel B). Analysis of these data revealed a main effect of treatment [F(2,22)=8.5, P<0.003] accounted for by a significant decrease in intake following Tat-RACK1 treatment (P<0.003), but not Tat-RACK1+K252a (P>0.05), as compared to intake after vehicle injection. These results suggest that the RACK1/BDNF is a homeostatic pathway that plays a role in regulating the behavioral effects of ethanol.

Discussion

We found that acute exposure to ethanol increases neuronal expression of BDNF via RACK1, and that increasing the protein levels of RACK1 in the brain increases the levels of BDNF. In addition, we found that the oral self-administration of ethanol increases BDNF expression in the striatum. We further found that decreasing the levels of BDNF or the inhibition of the BDNF pathway increases ethanol consumption as well as ethanol-induced place preference and sensitization. Conversely, increasing BDNF expression, either by ethanol exposure or via increasing the protein levels of RACK1, decreases ethanol consumption and sensitization.

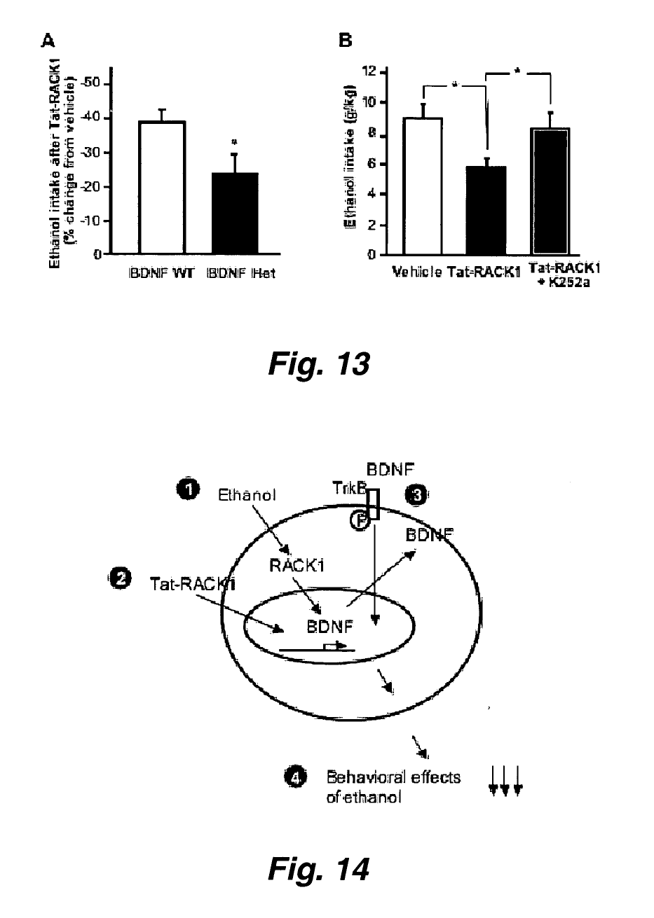

Based on these results we present the following model (FIG. 14). Acute or intermittent exposure to ethanol results in the induction of BDNF mRNA expression via nuclear RACK1 (FIG. 14-1). Increases in BDNF expression can also be achieved via the transduction of recombinant Tat-RACK1 (FIG. 14-2). Secreted BDNF then activates the BDNF signaling pathway (FIG. 14-3) to negatively regulate the behavioral effects of ethanol, including intake (FIG. 14-4).

The first step in the cascade is the nuclear translocation of RACK1 upon exposure to ethanol. The molecular mechanism that mediates the translocation of RACK1 to the nucleus in response to ethanol has been studied. Previously, we found that that ethanol induction of RACK1 translocation to the nucleus is mediated via the activation of the cAMP/PKA pathway (Ron etal. (2000) FASEB J. 14: 2303-2314; He et al. (2002) Mol. Pharm. 62: 272-280). Activation of the cAMP/PKA pathway by ethanol has been clearly established (Diamond and Gordon, (1997) Physiol. Rev. 77: 1-20). Interestingly, ethanol is not the only stimulus that can induce RACK1 nuclear translocation; stimulation of the PAC1 receptor by the neuropeptide, PACAP, also causes the translocation of RACK1 to the nucleus and this translocation is cAMP/PKA-dependent (Yaka et al. (2003) J. Biol. Chem. 278: 9630-9638). Thus the induction of RACK1 translocation via cAMP is not ethanol-specific BDNF heterozygote mice (BDNF±) showed a greater place preference to ethanol and greater ethanol self-administration after a deprivation period. Hence, the effects of ethanol were increased in two different animal models that are used to measure the reinforcing/rewarding effects of ethanol (place preference and self-administration). In addition, repeated ethanol treatment induced a greater degree of locomotor sensitization in BDNF±mice than in wildtype mice suggesting that the ethanol-induced plasticity that underlies sensitization occurs more readily in the heterozygote mice. These findings indicate that ethanol's effects are enhanced in the BDNF±mice. Conversely, reduction of BDNF signaling with the Trk receptor kinase antagonist, K252a, increased ethanol self-administration. Finally and importantly, ethanol consumption itself increases striatal expression of BDNF. These findings collectively indicate that endogenous BDNF systems normally act to inhibit some behavioral effects of ethanol, and that ethanol itself initiates this process.

An opposite pattern of results was obtained following Tat-RACK1 treatment. Tat-RACK1 injection reduced ethanol self-administration and blocked ethanol-induced behavioral sensitization. Our findings in dissociated neurons indicate that RACK1 increases BDNF expression and secretion. Our animal studies also provided evidence that the effects of RACK1 in vivo are mediated by BDNF: Tat-RACK1 injection increased striatal BDNF, the effects of Tat-RACK1 were reduced in BDNF±mice, and the Trk receptor kinase inhibitor, K252a, blocked the effect of Tat-RACK1 on ethanol intake.

Taken together the behavioral evidence suggests that activation of the RACK1/BDNF pathway negatively regulates ethanol effects, and thus this pathway may be acting in homeostatic fashion, i.e., functioning to maintain a stable behavioral state upon challenge with ethanol.

While exposure to ethanol is known to produce changes in signaling pathways that alter gene expression contributing to the development of alcohol addiction (Nestler (2001) Nat. Rev. Neurosci., 2: 119-128; Thibault et al. (2000) Mol. Pharmacol., 58: 1593-1600), our results suggest that some genes induced by exposure to ethanol are beneficial and may be part of a homeostatic pathway that controls behavioral adaptation to ethanol. Specifically, ethanol exposure may activate a signaling pathway that serves to control its own behavioral effects including consumption.

Our results are in agreement with Hensler et al. (2003) J. Neurochem. 85: 1139-1147, who reported that female BDNF heterozygous mice consume more ethanol than wildtype control mice. We believe the RACK1/BDNF pathway is shared by other drugs of abuse or naturally rewarding substances. We found that increasing brain levels of Tat-RACK1 by exogenous administration slightly decreased preference for a sweet solution. This suggests an intriguing means to control general processes of reward-seeking behavior through pharmacological intervention in the RACK1-BDNF pathway.

Interestingly, while we find that increases in BDNF levels are associated with reduction in ethanol's behavioral effects, some studies of the regulation of cocaine's effects by BDNF have found that increases in BDNF are associated with increases in cocaine-related behaviors. For example, Horger et al. found that infusion of BDNF into the NAc or the VTA enhances the acute responses to cocaine and enhances the development of sensitization to repeated administration of cocaine (Horger et al. (1999) J. Neurosci. 19: 4110-4122).

More recently, BDNF±mice have been reported to be less sensitive to cocaine's effects on locomotion and CPP (Hall et al. (2003) Neuropsychopharmacology 28: 1485-1490). In addition, Grimm et al. (2003) J. Neurosci. 23: 742-747, report that cue-induced reinstatement to cocaine increases with increasing length of withdrawal from cocaine, and increases in BDNF protein levels in the VTA, NAc and amygdala were also enhanced with increasing length of withdrawal, suggesting the involvement of BDNF in relapse to cocaine induced by conditioned stimuli. Taken together, these results might suggest fundamental differences in the regulation of the rewarding/reinforcing effects of cocaine and ethanol. However, other reports do suggest a potential inhibitory role for BDNF in the development of cocaine addiction. For example, it is well established that BDNF-regulated genes, such as dynorphin (Carlezon et al. (1998) Science 282: 2272-2275) and the D3 dopamine receptor (Pilla et al. (1999) Nature 400: 371-375; Xu et al. (1997) Neuron 19: 837-848), decrease the rewarding effects of cocaine. In addition, BDNF reverses molecular adaptations in the VTA observed after repeated cocaine administration (Berhow et al. (1995) Neuroscience 68: 969-979; Berhow et al. (1996) J. Neurosci. 16: 4707-4715). Thus, future studies of the mediation of the rewarding effects of cocaine, and other drugs of abuse, by the RACK1/BDNF pathway should shed light on these issues.

As mentioned above and, BDNF induces the expression of genes whose products have been demonstrated to reduce the reinforcing/rewarding effects of ethanol (Thiele et al. (1998) Nature 396: 366-369) or other drugs of abuse (Carlezon et al. (1998) Science 282: 2272-2275; Pilla et al. (1999) Nature 400: 371-375; Xu et al. (1997) Neuron 19: 837-848). For example, mice with a deletion of the gene for neuropeptide Y consume more ethanol than wild-type controls (Thiele et al. (1998) Nature 396: 366-369). In addition, studies suggest that the dopamine D3 receptor negatively regulates ethanol reward and consumption; administration of the D3 agonist 7-OH-DPAT decreases ethanol intake and preference in rats (Cohen et al. (1998) Psychopharmacology (Berl) 140: 478-485), while administration of the D3 antagonist U99194A enhances ethanol-induced conditioned place preference (Boyce and Risinger (2000) Brain Res., 880: 202-206). Activity of one or more of these gene products may be the mechanism for BDNF's reduction in ethanol's reinforcing/rewarding effects. In addition, BDNF has well described effects on dopaminergic and serotonergic release (Goggi et al. (2002) Brain Res. 941: 34-42; Martin-Iverson et al. (1994) J. Neurosci. 14: 1262-1270), and activation of selected serotonin receptor subtypes decreases ethanol intake (Tomkins et al. (2002) Phannacol. Biochem. Behav. 71: 735-744; Tomkins and O'Neill (2000) Phannacol. Biochem. Behav., 66: 129136; Wilson et al. (1998) Alcohol, 16: 249-270). It is also plausible that mechanisms that are involved in behaviors such as ethanol intake may depend upon synaptic plasticity, and studies suggest that BDNF plays an important role in synaptic functions including synaptic plasticity (Schuman (1999) Curr. Opin. Neurobiol. 9: 105-109). BDNF enhances presynaptic transmitter release (Patterson et al. (2001) Neuron 32: 123-140; Tyler and Pozzo-Miller (2001) J. Neurosci. 21: 4249-4258), and is involved in the induction of several forms of long-term potentiation (Korte et al. (1995) Proc Natl Acad Sci U.S.A., 92: 8856-8860; Kovalchuk et al. (2002) Science 295: 1729-1734). Recently, LTP induced by drugs of abuse, including ethanol (Saal et al. (2003) Neuron., 37: 577-582), has been described (Thomas and Malenka (2003) Phil. Trans: Biol. Sci., 358: 815-819). Whether BDNF modulates this drug-induced plasticity is not known, but it may be that this type of ethanol-induced plasticity may contribute to the effects described in the present studies. It is possible that activation of one or more of these mechanisms by BDNF within the brain circuitry that mediates ethanol's reinforcing effects may contribute to BDNF's effects on ethanol intake.

Our data suggest that the increase in BDNF mRNA in neurons is mediated by nuclear translocation of RACK1 upon acute treatment with ethanol. We also found that BDNF expression is reduced following chronic treatment with high concentrations of ethanol (FIG. 7B). The expression of BDNF mRNA in response to ethanol correlates with RACK1 nuclear compartmentalization; acute ethanol results in its translocation to the nucleus (He et al. (2002) Mol. Pharm. 62: 272-280; Ron et al. (2000) FASEB J. 14: 2303-2314), whereas prolonged ethanol incubation leads to the re-distribution of RACK1 out of the nucleus. These findings in cell culture suggest a breakdown of the homeostatic mechanism that functions to negatively regulate ethanol's effects. Specifically, it may be that during chronic sustained exposure to ethanol, the levels of BDNF are decreased, perhaps allowing the neuroadaptations that result in increases in drinking, as well as other behavioral phenotypes associated with alcohol addiction. The possible breakdown of homeostatic mechanisms that control ethanol intake is an important topic for future study.

In summary, our results suggest that the RACK1/BDNF pathway contributes to cellular and molecular processes that influence the motivation to consume alcohol. We propose that RACK1 and its target gene, BDNF, may be part of a homeostatic pathway that counteracts the adverse actions of alcohol that lead to phenotypes associated with addiction. We further hypothesize that when this pathway ceases to function properly, addiction may occur. These findings therefore open new avenues for the study and treatment of alcohol addiction.

Experimental Procedures

Reagents

K252a was purchased from Alamone Labs. Anti-RACK1, anti-TrkB antibodies were purchased from BD Transduction Laboratories and anti-HA antibodies were purchased from Roche Applied Science. Anti-phosphoTyr680/681Trk antibodies were purchased from Santa Cruz Biotechnology. TOTO-3 was purchased from Molecular Probes. Inc. Biotinylated donkey anti-rat antibodies were purchased from Jackson ImmunoResearch, ExtrAvidin peroxidase complex was purchased from Sigma, TSA™ Biotin System was purchased from Perkin-Elmer, and Avidin-FITC conjugate Biotin was purchased from Vector Labs. Protease inhibitor cocktail was purchased from Roche Applied Science. BDNF and NGF Emax ImmunoAssay System kits, Reverse Transcription System kit and PCR Master Mix were purchased from Promega Corporation. Primers for PCR were synthesized by BioSource International, Inc. Tat-fusion proteins were expressed in E. coli and purified as previously described (He et al. (2002) Mol. Pharm. 62: 272-280; Nagahara et al. (1998) Nat. Med. 4: 14491452). Tat-peptide (YGRKKRRQRRR, SEQ ID NO:7) was synthesized by Syn Pep. The purity of the peptide was greater than 90%, and the integrity was determined by Mass spectroscopy.

Animals

Male Sprague-Dawley and Long Evans rats were obtained from Harlan (Indianapolis, Ind.). C57B1/6 and BDNF±(J129ftm1Jae/C57BL6) mice were obtained from Jackson Laboratories (Bar Harbor, Me.). All rodents used in these studies were housed under a light:dark cycle of 12 hours, lights on at 6:00 a.m., with food and water available ad libitum. All procedures with animals in this report were approved by the Gallo Center Institutional Animal Care and Use Committee and were conducted in agreement with the Guide for the Care and Use of Laboratory Animals, National Research Council, 1996. Mixed pairs of BDNF±heterozygous hybrid male and female mice were used to generate heterozygote and wild type litter-mate control mice. Genotype of mice was determined by RT-PCR analysis of products derived from tail mRNA. F2 male mice used in the studies were 7-9 weeks of age at the beginning of experimental procedures.

Ethanol Self-Administration in Mice

Voluntary ethanol intake in singly-housed adult male C57B1/6 mice or BDNF+/+ and BDNF±mice was established by placing a bottle containing 10% ethanol in tap water (v/v) on the home cage next to an identical bottle filled with tap water for at least 1 month. Only subjects that consumed at least 40% of their total fluid volume as ethanol were included in these studies. All injections (PBS/10% glycerol vehicle, Tat-KEP27, K252a and/or Tat-RACK1) were conducted at 15:00 hours; ethanol and water intake were measured 18 hours later at 09:00 hours. Treatments were tested using a within-subjects design; subjects received one or two injections per week. Chemicals were administered in injection volumes of 1-3 ml per 100 g body weight. The effect of a deprivation period on consumption was tested by allowing mice continuous access to a bottle of 20% ethanol in tap water and a bottle of tap water on the home cage for 2 weeks. The ethanol bottle was removed for 2 weeks. Intake of 20% ethanol and of water was measured for 4 days after the ethanol was reintroduced.

Place Conditioning

The conditioned place preference (CPP) apparatus (Med Associates, St. Albans, Vt.) consisted of two visually distinctive conditioning compartments (one with white-colored walls and steel rod flooring and the other with black-colored walls and wire mesh flooring) connected by a smaller center compartment. The CPP procedure consisted of three phases: preconditioning (Day 1), conditioning (Days 2-9), and post-conditioning (Day 10). On Day 1, mice were placed into the center compartment and allowed free access to both conditioning compartments for 30 minutes. Mice were conditioned by pairing a 5-minute exposure of one compartment (either the black or the white) with ethanol (2 g/kg) injection, and of the other compartment with saline injection, on alternating days until subjects received 4 pairings each of ethanol and saline with their respective conditioning chambers. On Day 10, animals were tested for CPP by placing them in the center compartment and allowing free access to both conditioning compartments for 30 minutes.

Ethanol Self-Administration in Rats After Intracerebroventricular (i.c.v.) Microinjections

Adult male Long Evans rats were trained in the two-bottle choice ethanol self-administration procedure as described above for mice. Three weeks after ethanol exposure commenced, a 26 gauge cannula was placed into the right lateral ventricle (stereotaxic coordinates AP: −0.8, ML: −1.4, DV: −3.6, relative to bregma) of anesthetized subjects. Three weeks later, subjects received weekly i.c.v. microinjections of vehicle, Tat-RACK1, or the Tat-peptide (injection volume 5 ml; flow rate, 5 ml/5 minutes) at approximately 15:30, or 3.5 hours before lights out. Intake was measured 24 hours after treatment. All subjects received vehicle and Tat-RACK1 injections; a subset of subjects was also injected with the Tat-peptide. Cannula locations were verified histologically at the conclusion of the experiment.

Ethanol Sensitization

BDNF+/+ and BDNF±mice received daily saline injections for four days followed by placement within the 43 cm×43 cm locomotor activity chambers (Med Associates) for 15 minutes to determine the basal levels of locomotor activity. On Day 5, mice received 2 g/kg ethanol (v/v 20% solution, i.p.) and locomotor activity was measured for 15 minutes. Mice continued to receive daily ethanol (2.5 g/kg) injections once a day for 10 days in the home cage. On Day 16 mice were tested for the locomotor effects of a 2 g/kg ethanol challenge in the locomotor chambers for 15 minutes. For C57B1/6 mice, once daily saline habituation injections for three days were followed with saline or 2 g/kg ethanol administration as described above. Mice continued to receive either saline or ethanol (2.5 g/kg) injections daily. On Day 16, half the mice in each treatment group (saline and ethanol) were injected with 4 mg/kg Tat-RACK1, and half received vehicle. Locomotor effects of a 2 g/kg ethanol challenge were tested 3 hours later.

Saccharin and Quinine Preference Tests

Singly housed adult male C57B1/6 mice were allowed access to a bottle of tap water and a bottle of 0.033% saccharin in tap water or 0.015 mM quinine for 2 weeks, and the effects of i.p. Tat-RACK1 (4 mg/kg) were tested as described above.

Preparation of Slice and Brain Homogenates

Coronal striatal slices (250 mm) were prepared from 3-4 week old male Sprague-Dawley rats. Slices were maintained for at least 1 hour in artificial cerebrospinal fluid (αCSF) containing (in mM): 126 NaCl, 1.2 KCl, 1.2 NaH2PO4, 0.01 MgCl2, 2.4 CaCl2, 18 NaHCO3 and 11 glucose saturated with 95% O2/5% CO2 at 250 C. Following recovery, slices were treated as indicated in figure legends, homogenized in homogenization buffer containing 1% Triton X100, and (in mM) 320 Sucrose, 10 Tris-HCl, pH 7.4, 10 EDTA, 10 EGTA, and protease and phosphatase inhibitor cocktails. Brain homogenates were prepared after in vivo protein transduction. Adult C57B1/6 mice were injected i.p. with vehicle or 4 mg/kg of Tat-RACK1. Four hours later, brains were immediately removed and homogenized and protein extracts were prepared as described above.

Western Blot Analysis

Samples (50 mg) were resolved on a 10% SDS-PAGE and transferred to a nitrocellulose membrane. Membranes were probed with the appropriate antibodies and immunoreactivity was detected with enhanced chemilluminescence (Amersham Bioscience) and processed using the STORM system (Molecular Dynamic).

BDNF Expression in Primary Hippocampal Cultures

Sprague-Dawley rats (P0) were decapitated and hippocampi were dissected bilaterally. Cells were dissociated by enzyme digestion with papain followed by brief mechanical trituration and plated on poly-D-lysine treated flasks. Cells were plated (1×106 cells/chamber) and maintained in Neurobasal media supplemented with B27, penicillin, streptomycin and Glutamax-1 and maintained in culture for 3 weeks. Following treatment, total RNAs were isolated using Trizol reagent and reverse transcribed (RT) using the Reverse Transcription System kit. The RT products were used for analysis of the expression of BDNF and the control gene glycerol-3-phosphate dehydrogenase (GPDH) by polymerase chain reaction (PCR) described in (Yaka et al. (2003) J. Biol. Chem. 278: 9630-9638). The PCR products were photographed by Eagle Eye II (Stratagene), and quantified by using NIH Image 1.61.

BDNF Expression in Striatal Slices

Coronal striatal slices were prepared as described above. Following recovery, striatal slices were transferred to fresh aCSF containing 1 mM Tat-RACK1 and were incubated for the indicated time points. Following treatment, slices were extensively washed in aCSF and immediately frozen in liquid nitrogen for RT-PCR as described above for hippocampal cultures.

BDNF Expression In vivo

Adult male C57B1/6 mice were decapitated 3 hours after the start of the dark cycle, for mice with access to ethanol, or 6 hours following i.p. injection of vehicle or Tat-RACK1 (4 mg/kg), for naïve animals. Brains were rapidly removed and bilateral tissue punches of the striatum and PFC were frozen in liquid nitrogen. BDNF, NGF and GPDH expression were analyzed by RT-PCR as described above for hippocampal cultures. The primers for NGF were designed as follows:

(SEQ ID NO:8)

| | |
|---|---|
| Upstream | 5′-ACA CTC TGG ATC TAG ACT TCC AGG-3′; |
| and | |

(SEQ ID NO:9)

| | |
|---|---|
| Downstream | 5′-AGG CAA GTC AGC CTC TTC TTG TAG-3′. |

ELISA

BDNF or NGF secretion was measured in media from dissociated hippocampal neurons or from striatal slices using Emax ImmunoAssay according to (Balkowiec and Katz (2000) J. Neurosci., 20: 7417-7423)) and (Heaton et al. (2003) Brain Res. Dev. Brain Res. 140: 237-252), respectively. BDNF or NGF concentrations were interpolated from the standard curves (linear range of 7.8-500 pg/ml).

Immunohistochemistry

Primary hippocampal neurons were washed in cold PBS containing 0.1% Triton X-100, fixed in cold methanol and blocked in PBS containing 0.1% Triton X-100 and 3% Normal Goat Serum. lrmunostaining was performed with monoclonal monoclonal IgG HA antibodies (1:150). Staining was detected with anti FITC antibodies (1:500). Nuclei were detected with the nuclear marker TOTO-3 (1: 10000). Slides were viewed with a laser scanning confocal microscope (Zeiss LSM 510 Meta). Z-field images were processed by obtaining the middle Z field sections using LSM 5 Image Browse (Scion Corporation) and Adobe Photoshop (Adobe Systems Inc.). For immunocytochemistry used to detect the presence of Tat-RACK1 in the brain, C57 mice were injected i.p. with vehicle or 4 mg/kg Tat-RACK1. Animals were decapitated 4 hours post injection and brains were quickly removed and frozen at −50° C. in isopentane. Slide-mounted saggital striatal sections (16 mm) were immersed for 10 minutes in cold (−20° C.) mixture of 75% methanol and 25% acetone, re-hydrated, rinsed in PBS and incubated in 10% normal donkey serum. Rat monoclonal anti-HA antibody (1:2,000 in PBS) was applied overnight in a humid chamber. Sections were then rinsed in PBS, blocked in 2% normal donkey serum for 10 minutes, and incubated in biotinylated donkey anti-rat antibody (1:300 in PBS) for 2 hours, followed by incubation with the ExtrAvidin peroxidase complex (1:3000) for 2 hours and rinses in PBS. Slides were viewed with a laser scanning confocal microscope by using TSA™ Biotin System and Avidin-FITC conjugate (1:500).

Statistics

All mRNA and protein results were analyzed using Student's t-test. Behavioral data are depicted in figures as means±SEM and were analyzed using one- or two-way ANOVA, with repeated measures as appropriate. Significant main effects or interactions were followed by post-hoc tests using Student Newman-Keuls method. Significance for all tests was set at P<0.05.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of identifying an agent that mitigates one or more symptoms of substance abuse, said method comprising:
contacting a cell or tissue with a test agent; and
determining whether or not there is an increase in expression or activity of a GDNF pathway component, wherein an increase in expression or activity of a GDNF pathway or component, as compared to a control, indicates that said agent is an agent that mitigates a symptom of substance abuse.

2. The method of claim 1, wherein said GDNF pathway component is selected from the group consisting of GDNF, GFRα1, and RET.

3. The method of claim 1, wherein said determining comprises determining the association between GDNF and GFRα1.

4. The method of claim 1, wherein said determining comprises determining the phosphorylation of RET.

5. The method of claim 1, wherein said determining comprises determining the phosphorylation of RET.

6. The method of claim 1, wherein said determining comprises determining the expression level of a component selected from the group consisting of GDNF, GFRα1, or RET.

7. The method of claim 42, wherein said cell or tissue is a nerve cell or tissue.

8. The method of claim 42, wherein said cell or tissue is a cell in a brain tissue preparation

9. The method of claim 42, wherein said cell is a cell in culture.

10. The method of claim 9, wherein said cell is an SHSY5Y cell.

11. The method of claim 1, wherein said determining comprises a nucleic acid hybridization to determine an mRNA level.

12. The method of claim 11, wherein said determining comprises a method selected from the group consisting of a Northern blot, a Southern blot using DNA derived from an RNA encoding a protein in a GDNF pathway, an array hybridization, an affinity chromatography, an RT-PCR using an RNA encoding a protein in a GDNF pathway, and an in situ hybridization.

13. The method of claim 1, wherein said detecting comprises detecting a protein in a GDNF pathway.

14. The method of claim 51, wherein said detecting is via a method selected from the group consisting of capillary electrophoresis, a Western blot, mass spectroscopy, ELISA, immunochromatography, and immunohistochemistry.

15. The method of claim 1, wherein said control comprises a cell contacted with said test agent at a lower concentration.

16. The method of claim 1, wherein said control comprises a cell or tissue not contacted with said test agent.

17. The method of claim 1, wherein said test agent is not an antibody.

18. The method of claim 1, wherein said test agent is not a protein.

19. The method of claim 1, wherein said test agent is a small organic molecule.

20. The method of claim 1, wherein said, wherein said test agent is contacted to a cell containing the component.

21. The method of claim 20, wherein said cell is cultured ex vivo.

22. The method of claim 1, wherein said, wherein said test agent is administered to an animal comprising a cell containing the component.

23. A method of mitigating one or more symptoms of substance abuse in a mammal, said method comprising increasing the level, expression, or activity of GDNF in said mammal.

24. The method of claim 23, wherein said symptom is selected from the group consisting of intake (self-administration) of said substance of abuse, preference for said substance of abuse, relapse, and a symptom of withdrawal.

25. The method of claim 23, wherein said method does not comprise administering ibogaine.

26. The method of claim 23, wherein said method comprises increasing the expression or activity of GFRα1.

27. The method of claim 23, wherein said method comprises increasing the phosphorylation of RET.

28. The method of claim 23, wherein said method comprises administering an ibogaine analogue to said mammal.

29. The method of claim 23, wherein said method comprises administering GDNF to said mammal.

30. The method of claim 23, wherein said method comprises administering a GDNF mimetic to said mammal.

31. A method of mitigating one or more symptoms of substance abuse in a mammal, said method comprising: increasing activity of a GDNF pathway.

32. A method of mitigating one or more symptoms of substance abuse in a mammal, said method comprising: increasing the expression or activity of BDNF, RACK1, and/or the dopamine D3 receptor (D3R) in said mammal.

33. The method of claim 32, wherein said method comprises increasing the expression or activity of BDNF.

34. The method of claim 32, wherein said method comprises increasing the expression or activity of RACK1.

35. The method of claim 32, wherein said method comprises increasing the expression or activity of the dopamine D3 receptor.

36. The method of claim 32, wherein said method comprises administering RAC1 or an analogue or mimetic thereof.

37. The method of claim 36, wherein said RAC1 is administered as a fusion protein tat-RAC1.

38. The method of claim 32, wherein said symptom is intake (self-administration) of said substance of abuse.

39. The method of claim 32, wherein said symptom is preference for said substance of abuse.

40. The method of claim 32, wherein said symptom is relapse.

41. A method of mitigating one or more symptoms of substance abuse in a mammal, said method comprising: increasing activity of a BDNF pathway.

42. A method of identifying an agent that mitigates one or more symptoms of substance abuse, said method comprising:
contacting a cell or tissue with a test agent; and
determining whether or not there is an increase in expression or activity of a component of a BDNF pathway, wherein an increase in expression or activity of a component of a BDNF pathway, as compared to a control, indicates that said agent is an agent that mitigates a symptom of substance abuse.

43. The method of claim 42, wherein said determining comprises determining an increase in expression or activity of BDNF.

44. The method of claim 42, wherein said determining comprises determining an increase in expression or activity of RACK1.

45. The method of claim 42, wherein said cell or tissue is a nerve cell or tissue.

46. The method of claim 42, wherein said cell or tissue is a cell in a brain slice preparation

47. The method of claim 42, wherein said cell is a cell in culture.

48. The method of claim 42, wherein said determining comprises determining the level of phosphorylation of TrkB.

49. The method of claim 42, wherein said determining comprises a nucleic acid hybridization to determine an mRNA level.

50. The method of claim 49, wherein said detecting comprises a method selected from the group consisting of a Northern blot, a Southern blot using DNA derived from an RNA encoding a protein in BDNF pathway, an array hybridization, an affinity chromatography, and an in situ hybridization.

51. The method of claim 42, wherein said detecting comprises detecting a protein in a BDNF pathway.

52. The method of claim 51, wherein said detecting is via a method selected from the group consisting of capillary electrophoresis, a Western blot, mass spectroscopy, ELISA, immunochromatography, and immunohistochemistry.

53. The method of claim 42, wherein said control comprises a cell contacted with said test agent at a lower concentration.

54. The method of claim 42, wherein said control comprises a cell or tissue not contacted with said test agent.

55. The method of claim 42, wherein said test agent is not an antibody.

56. The method of claim 42, wherein said test agent is not a protein.

57. The method of claim 42, wherein said test agent is a small organic molecule.

58. A method of prescreening for an agent that modulates an organism's response to a substance of abuse, said method comprising
i) contacting a component of a GDNF pathway or a BDNF pathway or a nucleic acid encoding said component with a test agent; and
ii) detecting specific binding of said test agent to said component or to said nucleic acid, wherein specific binding of said test agent to said component or to said nucleic acid indicates that said agent is likely to mitigate said organism's response to a substance of abuse.

59. The method of claim 58, further comprising recording test agents that specifically bind to said nucleic acid or to said component in a database of candidate agents that alter an organism's response to a substance of abuse.

60. The method of claim 58, wherein said component is not a D3 receptor.

61. The method of claim 58, wherein said test agent is not an antibody.

62. The method of claim 58, wherein said test agent is not a protein.

63. The method of claim 58, wherein said test agent is not a nucleic acid.

64. The method of claim 58, wherein said test agent is a small organic molecule.

65. The method of claim 58, wherein said, wherein said detecting comprises detecting specific binding of said test agent to said nucleic acid.

66. The method of claim 65, wherein said binding is detected using a method selected from the group consisting of a Northern blot, a Southern blot using DNA derived from an GDNF or BDNF pathway RNA, an array hybridization, an affinity chromatography, and an in situ hybridization.

67. The method of claim 58, wherein said detecting comprises detecting specific binding of said test agent to said component.

68. The method of claim 67, wherein said, wherein said detecting is via a method selected from the group consisting of capillary electrophoresis, a Western blot, mass spectroscopy, ELISA, immunochromatography, and immunohistochemistry.

69. The method of claim 58, wherein said, wherein said test agent is contacted directly to the component or to the nucleic acid.

70. The method of claim 58, wherein said, wherein said test agent is contacted to a cell containing the component.

71. The method of claim 70, wherein said cell is cultured ex vivo.

72. The method of claim 58, wherein said, wherein said test agent is administered to an animal comprising a cell containing the component.

73. A kit for mitigating one or more symptoms of substance abuse, said kit comprising:
a container containing an agent that increases expression and/or activity of a component of a GDNF pathway and/or a BDNF pathway; and
instructional materials teaching the use of said agent to modulate an organism's response to a substance of abuse or to withdrawal therefrom.
